(19) United States
(12) Patent Application Publication
(10) Pub. No.: US 2022/0253671 A1
(43) Pub. Date: Aug. 11, 2022

(54) GRAPH NEURAL DIFFUSION

(71) Applicant: Twitter, Inc., San Francisco, CA (US)

(72) Inventors: Ben Chamberlain, London (GB); Cristian Bodnar, San Francisco, CA (US); Francesco Di Giovanni, San Francisco, CA (US); Michael M. Bronstein, London (GB)

(21) Appl. No.: 17/650,219

(22) Filed: Feb. 7, 2022

Related U.S. Application Data
(60) Provisional application No. 63/199,980, filed on Feb. 5, 2021.
(60) Provisional application No. 63/262,519, filed on Oct. 14, 2021.
(60) Provisional application No. 63/264,154, filed on Nov. 16, 2021.

(51) Int. Cl. G06N 3/04; G06N 3/08; G06F 17/16

(52) U.S. Cl. CPC G06N 3/04; G06F 17/16; G06N 3/08

(57) ABSTRACT

Improved graph neural networks (GNNs) include defining a GNN architecture based on a discretized non-Euclidean diffusion partial differential equation (PDE) such that evolution of feature coordinates represents message passing layers in a GNN and evolution of positional coordinates represents graph rewiring. The GNN being based on both position and feature coordinates has their evolution derived from Beltrami flow. The Beltrami flow is modeled using a Laplace-Beltrami operator, which is a generalization of the Laplace operator to functions defined on submanifolds in Euclidean space and on Riemannian manifolds. The discretization of the spatial component of the Beltrami flow offers a principled view on positional encoding and graph rewiring, whereas the discretization of the temporal component can replace GNN layers with more flexible adaptive numerical schemes. Based on this model, Beltrami Neural Diffusion (BLEND) that generalizes a broad range of GNN architectures is introduced; BLEND shows state-of-the-art performance on many benchmarks.

### RELATED APPLICATIONS

This application is a non-provisional of, and claims priority to, U.S. Provisional Application No. 63/199,980, filed on Feb. 5, 2021, entitled “GRAPH NEURAL PDES,” the disclosure of which is incorporated herein in its entirety.

This application is a non-provisional of, and claims priority to, U.S. Provisional Application No. 63/262,519, filed on Oct. 14, 2021, entitled “GRAPH NEURAL PDES,” the disclosure of which is incorporated herein in its entirety.

This application is a non-provisional of, and claims priority to, U.S. Provisional Application No. 63/264,154, filed on Nov. 16, 2021, entitled “GRAPH DIFFUSION AND EMBEDDING ENERGIES,” the disclosure of which is incorporated herein in its entirety.

### TECHNICAL FIELD

This description relates to generation and/or use of graph neural networks (GNNs) for a social network and/or other applications within a computer system.

### BACKGROUND

Graphs are a kind of data structure which models a set of objects (nodes) and their relationships (edges). For example, graphs are used to analyze and draw conclusions about relationships in a wide variety of applications. A graph neural network (GNN) is a type of neural network which operates on graphs. Some computer-implemented tasks implemented by GNNs on graphs include node classification, link prediction, and/or clustering. However, some conventional GNNs are susceptible to a bottleneck when aggregating messages across a long path (e.g., between distant nodes), and the bottleneck may cause over-squashing of information into fixed-size vectors.

### SUMMARY

Techniques discussed herein overcome technical problems involving graph neural networks (GNNs), namely bottlenecking and oversmoothing phenomena that may result in lost messages between sufficiently distant nodes. Accordingly, applications of the techniques discussed herein may result in a GNN model that is not limited by bottlenecking (or bottlenecking is reduced) and hence provide more accurate message-passing, which may improve the accuracy of predicting information from a graph. Also, the GNN model discussed herein is executable by a computing system, and the structure of the GNN model may improve the performance of the computing system itself. For example, the GNN model may reduce the amount of computing resources (e.g., CPU, memory) of the computing system used to execute the GNN model and/or increase the speed at which the GNN model operates on a graph. In some examples, the structure of the GNN model discussed herein may provide a more lightweight model (in terms of computing resources) while increasing the accuracy of operations on graph data.

In one general aspect, a method can include obtaining graph data representing a first graph, the first graph representing a social network and having (i) a plurality of nodes representing users of the social network and (ii) a plurality of edges connecting pairs of nodes of the plurality of nodes and representing connections between the users of the social network, each of the plurality of nodes having a respective set of feature coordinates representing a set of features and a set of positional coordinates representing a set of positions, the set of feature coordinates and the set of positional coordinates defining a first embedding. The method can also include inputting the graph data into a graph neural network (GNN) model. The method can further include producing, as output of the GNN model, a second embedding vector for each of the set of nodes via a second learnable function of the respective set of features and the respective set of positions of each of the plurality of nodes, the second embedding vector for at least one of the set of nodes resulting in a labeling of the users of the social network represented by the plurality of nodes and a rewiring of the graph.

The details of one or more implementations are set forth in the accompanying drawings and the description below. Other features will be apparent from the description and drawings, and from the claims.

### BRIEF DESCRIPTION OF THE DRAWINGS

FIG. 1A is a diagram that illustrates an example computing system having a graph neural network (GNN) according to an aspect.

FIG. 1B is a diagram that illustrates an example GNN general design pipeline for designing a GNN model according to an aspect.

FIG. 2 is a diagram that illustrates an example electronic environment having a GNN according to an aspect.

FIG. 3 is a diagram that illustrates an example messaging system having a GNN according to an aspect.

FIG. 4 illustrates a flow chart illustrating an example method of performing an implementation of the improved techniques.

FIG. 5 illustrates a flow chart illustrating an example method of performing an implementation of the improved techniques.

FIG. 6 is a diagram illustrating two example interpretations of Beltrami flow.

FIG. 7 is a diagram that illustrates an example Beltrami flow in a hyperbolic coordinate system.

### DETAILED DESCRIPTION

The improvements detailed herein are directed to defining a graph neural network (GNN) architecture based on a discretized non-Euclidean diffusion partial differential equation (PDE). The PDE governs an evolution of feature coordinates associated with each node of a graph, representing message passing layers in a GNN. The PDE also governs an evolution of positional coordinates associated with each node of the graph, representing a particular wiring of the graph. By utilizing a discretization of the PDE, a GNN that passes messages between nodes efficiently and accurately.

In some examples, a graph may represent a social network. In some examples, the nodes may represent users, and edges may represent connections between users. In this case, the feature coordinates for a node may represent data about the user represented by the node, whose updates are communicated between nodes via message passing layers. A GNN architecture governed by the discretized diffusion PDE, rather than an arbitrarily-configured GNN, governs the layers and the message passing between the layers. The advantage of such a GNN architecture is that the GNN overcomes bottlenecks and over smoothing and accordingly passes messages between nodes separated by a large distance. Messages representing changes to the feature coordinates indicate how updates to user information are propagated through the graph. Updates to the positional coordinates automatically indicate a rewiring of the graph. Such GNN architectures may have applications in link prediction, identifying malicious actors in the social network, and generating suggestions for who users should follow.

GNNs are machine learning-based methods that operate on graph domains. GNNs have been shown to improve efficiency in many machine learning analyses such as node classification, link prediction, and/or graph classification. Some design pipelines of a GNN model may include four steps: (1) find graph structure, (2) specify graph type and scale, (3) design loss function and (4) build model using computational modules.

A graph structure may typically be determined from two scenarios: structural scenarios and non-structural scenarios. In structural scenarios, the graph structure is explicit in the applications, such as applications on molecules, physical systems, knowledge graphs and the like. In non-structural scenarios, graphs are implicit so that one first builds the graph from a task, i.e., building a fully-connected “word” graph for text or building a scene graph for an image. After the graph structure is obtained, an optimal GNN model may be determined/selected from the obtained graph.

For graph types, one finds out the graph types after obtaining the graph structure. Graph types include directed or undirected, homogeneous or heterogeneous, and static or dynamic. Edges in directed graphs are all directed from one node to another. Nodes and edges in homogeneous graphs have the same types, while nodes and edges have different types in heterogeneous graphs. In dynamic graphs, the graph topology varies with time.

Some social networks may be represented by graphs. A graph G is a data structure that has a set of nodes or vertices V and a set of edges E; each of the set of edges represents a relationship between two of the set of nodes of the graph G. For a social network, each of the set of nodes V may represent a person in the social network, and each of the set of edges E may represent a connection between two people in the social network. Each of the set of nodes V may be associated with a feature vector representing features of the node and a position vector representing a position of the node within the graph.

In the context of social networks, graphs may be analyzed in order to perform node classification, link prediction, and/or clustering. Some graph analysis is performed by graph neural networks (GNNs). GNNs are deep learning-based methods that operate on graphs.

In the context of social networks, a GNN may be used to perform classifications of users within the network. For example, a GNN may be trained to determine whether a user in a social network is a malicious actor, e.g., a spoof account, a provider of misinformation, etc. Such a malicious actor may be identified based on the node degree, specific connections to other users, or particular combinations of features.

GNNs may follow a message passing paradigm which uses learnable nonlinear functions to propagate information (e.g., messages) on a graph. Assuming the graph G is a simple, undirected, and connected graph, and define (i,j)∈E⇔i˜j. The focus here is on the unweighted case, although the theory extends to the weighted setting as well. Denote the adjacency matrix by A and let Ã=A+I be an augmented adjacency matrix augmented with self-loops. Similarly, define \tilde{D}=D+I where D is a diagonal degree matrix, and let

$$\hat{A} = {D}^{- \frac{1}{2}} \tilde{A} {D}^{- \frac{1}{2}}$$

be a normalized augmented adjacency matrix. Given i∈V, denote its degree as dᵢ (without accounting for self-loops) and define

Sᵣ(i)≡{j∈V:d_{G}(i,j)=r}

and

Bᵣ(i)≡{j∈V:d_{G}(i,j)≤r},

where d_{G} is a minimum-walk (geodesic) distance on the graph G and r∈ [symbol] . The set Bᵣ(i) represents a receptive field of an r-layer message passing neural network at node i.

Assume the graph G is equipped with node features X∈ [symbol] ^{n×p}^{₀}, where xᵢ∈ [symbol] ^{p}^{₀} is the feature vector at node i∈{1, 2, . . . n=|V|}. Denote hᵢ^{(l)}∈ [symbol] ^{p}^{ₗ} as a representation of node i at layer l≥0, with hᵢ⁽⁰⁾=xᵢ. Given a family of message functions ψₗ: [symbol] ^{p}^{ₗ}× [symbol] ^{p}^{ₗ}→ [symbol] ^{p′}^{ₗ} and update functions ϕₗ: [symbol] ^{p}^{ₗ}× [symbol] ^{p′}^{ₗ}→ [symbol] ^{p}^{ₗ₊₁}, the (l+1)-th layer output of a conventional GNN may be expressed as follows:

$${h}_{i}^{( l + 1 )} = {ϕ}_{l} ( {h}_{i}^{( l )} , {\sum }_{j = 1}^{n} {\hat{a}}_{ij} {ψ}_{l} ( {h}_{i}^{( l )} , {h}_{j}^{( l )} ) ) . \qquad$$

Here, the augmented normalized adjacency matrix is used to propagate messages from each node to its neighbors, which may lead to a degree normalization of the message functions ψₗ.

Conventional GNNs may perform poorly in situations when the learned task requires long-range dependencies and at the same time the structure of the graph results in exponentially many long-range neighboring nodes. Some graph learning problems have long-range dependencies when the output of a conventional GNN depends on representations of distant nodes interacting with each other. If long-range dependencies are present, messages coming from non-adjacent nodes may need to be propagated across the network without being too distorted. In many cases, however (e.g., in ‘small-world’ graphs such as social networks), the size of the receptive field Bᵣ(i) grows exponentially with r. If this occurs, representations of exponentially many neighboring nodes may need to be compressed into fixed-size vectors to propagate messages to node i, causing a phenomenon referred to as over-squashing of information. Those structural properties of the graph that lead to over-squashing are referred to as a bottleneck.

The hidden feature hᵢ^{(l)}=hᵢ^{(l)}(x₁, x₂, . . . , xₙ) computed using a conventional GNN with l layers is a differentiable function of the input node features {x₁, x₂, . . . , xₙ} as long as the update and message functions ϕₗ and ψₗ are differentiable. The over-squashing of information can then be understood in terms of one node representation hᵢ^{(l)} failing to be affected by some input feature xₛ of node s at distance r from node i (i.e., r edges between node s and node i). Accordingly, the Jacobian ∂hᵢ^{(r+1)}/∂xₛ may be used as an explicit and formal way of assessing the over-squashing effect: assuming a conventional GNN defined as above and let i, s∈V with s∈Sᵣ(i), if |∇ϕₗ|≤α and |∇ψₗ|≤β for 0≤l≤r, then |∂hᵢ^{(r+1)}/∂xₛ|≤(αβ)^{r+1}(Â^{r+1})ᵢₛ. As a corollary, if hᵢ^{(l+1)}=Σ_{i˜j}ψₗ(hⱼ^{(l)}, then hᵢ^{(l+1)} only depends on nodes that can be reached via walks of length exactly l+1. If ϕₗ and ψₗ have bounded derivatives, then the propagation of messages is controlled by a suitable power of Â. For example, if d_{G}(i,s)=r+1 and the sub-graph induced on Bᵣ₊₁(i) is a binary tree, then (Âr+1)ᵢₛ=2⁻¹₃^{-r} which gives an exponential decay of the node dependence on input features at distance r.

In contrast to the above-described conventional GNNs, which may suffer from inefficiencies and inaccuracies due to the over-squashing effect, improved techniques include defining a GNN architecture based on a discretized non-Euclidean diffusion partial differential equation (PDE) such that evolution of feature coordinates represents message passing layers in a GNN model and evolution of positional coordinates represents graph rewiring. The GNN models being based on both position and feature coordinates have their evolution derived from Beltrami flow. The Beltrami flow is modeled using a Laplace-Beltrami operator, which is a generalization of the Laplace operator to functions defined on submanifolds in Euclidean space and, even more generally, on Riemannian manifolds. The discretization of the spatial component of the Beltrami flow offers a principled view on positional encoding and graph rewiring, whereas the discretization of the temporal component can replace GNN layers with more flexible adaptive numerical schemes. Based on this model, Beltrami Neural Diffusion (BLEND) that generalizes a broad range of GNN architectures is introduced; BLEND shows state-of-the-art performance on many popular benchmarks. In a broader perspective, this approach explores new tools from PDEs and differential geometry that are less well known in the graph machine learning community.

In the above-described improved techniques, the PDE, the discretization of which defines the GNN architecture governs the behavior of the feature coordinates and the positional coordinates over space and time. In other words, each of the feature coordinates and the positional coordinates satisfy the PDE. More generally, one may form vector spaces based on combinations of the vector space of the feature coordinates and the vector space of the positional coordinates that satisfy the PDE. In one example, an embedding vector resulting from a concatenation of the positional coordinates and the feature coordinates satisfies the PDE. In another example, a cellular sheaf including the vector spaces of the positional coordinates and feature coordinates satisfies the PDE.

A GNN architecture is defined by connections between input and output graph layers; a GNN model is defined by a process by which an input graph at the input layer becomes an output graph at the output layer. Accordingly, the GNN architecture may be at least partially defined by message-passing (hidden) layers between the input and output layers; the GNN model is defined by the message-passing itself which is governed by the discretized PDE. Because the number of hidden layers used in the GNN model is based on a discretization scheme for the PDE, the GNN architecture is based on the discretization scheme.

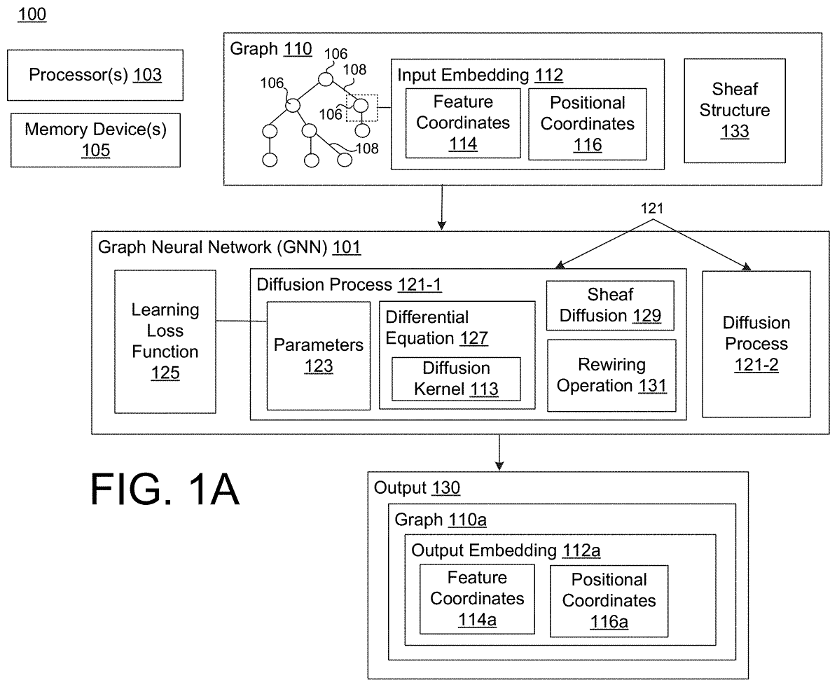

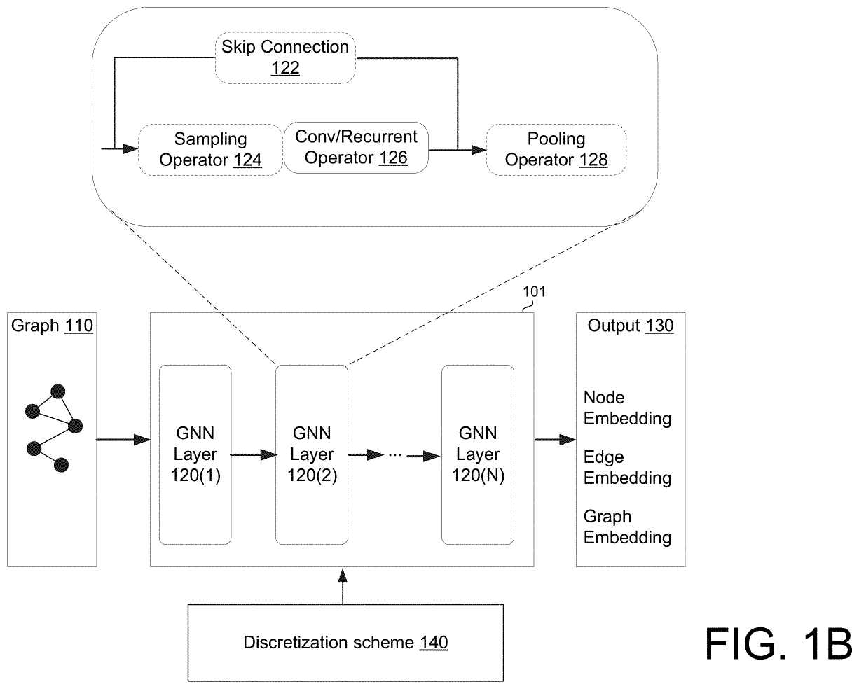

FIG. 1A illustrates a computing system 100 that utilizes a graph neural network (GNN) model 101 according to an aspect. FIG. 1B illustrates an aspect of the GNN model 101 according to an aspect. The computing system 100 may be any type of computing system having one or more processors 103 and one or more memory devices 105 configured to execute the GNN model 101. The techniques discussed herein may be applied to any application having a graph representation. For example, the computing system 100 uses the GNN model 101 in the modeling of real-world entities and their interactions as graphs 110. The GNN model 101 may receive a graph 110 and generate an output 130. The output 130 may represent a prediction, which may widely vary depending on the application. In some examples, the GNN model 101 is configured to execute node classification on the graph 110 in which the output 130 may represent a labeling (e.g., output labels) of at least a portion of nodes 106 of the graph 110. In some examples, the GNN model 101 is configured to execute link prediction on the graph 110 in which the output 130 may represent predicted connections in the graph 110. In some examples, the GNN model 101 is configured to execute a clustering operation on the graph 110 in which the output 130 may represent one or more groups of nodes 106 and/or edges 108.

In some examples, the computing system 100 includes a messaging system (e.g., a social media messaging system). The messaging system may use the GNN model 101 to recommend information to users. In some examples, the messaging system may use the GNN model 101 to recommend messages, topics, interests, user accounts to establish a connection (e.g., follow, friend, etc.), news articles, and/or promoted information such as promoted messages or profiles. In some examples, the messaging system may use the GNN model 101 to classify information posted on the messaging platform (e.g., messages, hashtags, terms used within messages), information determined or detected by the messaging platform, and/or classify users of the messaging platform such as labeling user accounts that violate one or more terms of the messaging platform (e.g., malicious actors posting toxic, abusive, an/or spam messages). In some examples, the messaging platform may use the GNN model 101 to cluster information posted to the messaging platform and/or users of the messaging platform.

In some examples, the computing system 100 includes an image processing system (e.g., computer vision). For example, the image processing system may use the GNN model 101 for image processing such as image classification and/or the edge-preserving denoising of images. In some examples, the computing system 100 includes a text processing system, where the text processing system may use the GNN model 101 for text processing such as natural language processing and computer-implemented reading comprehension (e.g., given a text passage, the GNN model 101 may provide one or more answers by consolidating information in the passage). However, the computing system 100 may use the GNN model 101 for a wide variety of use cases, such as travel time predictions, product/service recommendation systems, self-driving vehicles, robotics, combinatorial optimization (CO) problems,

In some examples, the computing system 100 uses the GNN model 101 for molecular or chemical structures, including computer-assisted drug design. In some examples, the computing system 100 may represent interactions between particles or molecules as a graph and use the GNN model 101 to predict the properties of such systems. In some examples, the computing system 100 uses the GNN model 101 for computer-assisted drug design. For example, drug discovery may start from the initial stage of drug discovery where it identifies certain groups of molecules that are likely to become a drug. Then, it goes through several steps to eliminate unsuitable molecules and finally tests them in real life. In some examples, absorption, distribution, metabolism, and/or excretion (ADME) properties may be obtained during the drug discovery stage. Drug discovery may be modelled as an optimization problem, where the ADME properties are predicted, and molecules are selected to increase the likelihood of developing a safe drug. In some examples, the computing system 100 uses the GNN model 101 to predict the properties (e.g., ADME properties) of a new molecule. In some examples, to apply the GNN model 101 to molecular structures, the molecule is transformed into a numerical representation that can be understood by the GNN model 101. In some examples, the GNN model 101 receives information about each individual atom and information about neighboring atoms in the form of a feature vector. In some examples, the feature vector may include information about the atomic number, number of valence electrons, or number of single bonds.

The GNN model 101 may include a messaging passing neural network. In some examples, a GNN model 101 is a neural model that captures the dependence of a graph 110 via messaging passing between nodes 106 of the graph 110. In other words, the GNN model 101 uses a message passing paradigm, in which feature coordinates 114 representing a first set of features of a respective node 106 are propagated on the graph 110.

In some conventional GNNs, information may become distorted as the feature coordinates 114 are propagated to distant nodes 106 (e.g., non-adjacent nodes 106), which can decrease the efficiency for tasks (e.g., node classification, link prediction, clustering) that rely upon long-distance interactions. In some examples, a second node is distant from a first node when the second node is at least a threshold distance (e.g., threshold distance r). In some examples, this phenomenon may be referred to as over-squashing, which may be attributed to graph bottlenecks where the number of k-hop neighbor nodes 106 grows rapidly with k. However, the architecture of the GNN model 101 as discussed herein may improve the performance of the GNN model 101 itself by reducing the graph bottlenecks and/or reducing the distortion of information from distant nodes, which can minimize or alleviate over-squashing (thereby improving the prediction efficiency of the GNN model 101). Also, the architecture of the GNN model 101 may reduce the amount of computing resources (e.g., processor(s) 103, memory device(s) 105) used to execute the GNN model 101 and/or increase the speed at which the GNN model 101 operates on a graph 110. In some examples, the architecture of the GNN model 101 discussed herein may provide a lightweight model (in terms of computing resources) while increasing the accuracy of operating on the graph 110.

The computing system 100 may obtain graph data representing at least a portion of a graph 110. The graph 110 includes a plurality of nodes 106 representing entities and a plurality of edges 108 representing connections between the nodes 106. For example, each node 106 represents a separate entity. An edge 108 may represent a connection between a pair of nodes 106. The type of entity depends widely on the application of the computing system 100. In a messaging system, the plurality of nodes 106 may represent users (e.g., user accounts) of a messaging platform, and the edges 108 may represent relationships (e.g., friends, follow) between the users.

The graph 110 may have a graph structure such as a structural graph or a non-structural graph. In structural scenarios, the graph structure is explicit in the applications, such as applications on molecules, physical systems, knowledge graphs and the like. In non-structural scenarios, graph structure is implicit in the sense that the graph is built from a task, e.g., building a fully connected “word” graph for text or building a scene graph for an image.

The graph 110 may have a graph type. In some examples, the graph 110 includes a directed graph. In some examples, the graph 110 includes an undirected graph. In some examples, the graph 110 includes a homogenous graph. In some examples, the graph 110 includes a heterogeneous graph. In some examples, the graph 110 includes a static graph (e.g., time-independent). In some examples, the graph 110 includes a dynamic graph (e.g., time-dependent). Edges 108 in directed graphs are all directed from one node 106 to another node 106. Nodes 106 and edges 108 in homogeneous graphs have the same types, while nodes 106 and edges 108 have different types in heterogeneous graphs. In dynamic graphs, the graph topology varies with time.

Each of at least a portion of the nodes 106 includes (or is associated with) feature coordinates 114 representing a first set of features. In some examples, each of the nodes 106 in the graph 110 (or a portion thereof) includes (or is associated with) feature coordinates 114. The feature coordinates 114 may be a numerical representation of a set of features. In some examples, the feature coordinates 114 may be referred to as features or feature vector(s). In some examples, the feature coordinates 114 may include one or more feature vectors describing a first set of features. The type of features depends on the application of the computing system 100. In the case of a messaging platform, for a respective node 106, the set of features may include information about a user account (e.g., profile information, interests, etc.) and/or information about the user's use on the platform (e.g., messages posted, engagements, re-shares, replies, etc.).

Each of at least a portion of the nodes 106 includes (or is associated with) positional coordinates 116 representing a position of a respective node 106 within the graph 110. In some examples, each of the nodes 106 in the graph 110 (or a portion thereof) includes (or is associated with) positional coordinates 116. In some examples, the positional coordinates 116 represent a numerical representation of the position or location of a respective node 106 within the graph 110. In some examples, the position is a location in three-dimensional (3D) space. In some examples, the positional coordinates 116 represent vertex positions within the graph 110. In some examples, the positional coordinates 116 include positional data in a non-Euclidean coordinate system with a metric. In some examples, the non-Euclidean coordinate system is a hyperbolic coordinate system.

In some examples, the graph 110 defines an input embedding 112, where the input embedding 112 includes the feature coordinates 114 and the positional coordinates 116 for each node 106 of the graph 110 (or at least some of the nodes 106 of the graph 110). For example, the feature coordinates 114 and the positional coordinates 116 may be combined (e.g., concatenated) to form the input embedding 112. In some examples, the input embedding 112 is referred to as an embedding vector (or input embedding vector). In some examples, the input embedding 112 is defined in a metric space having an input metric. In some examples, the input metric is non-Euclidean. In some examples, the metric evolves with time. In some examples, the graph 110 does not define an embedding.

In some examples, the graph 110 includes a sheaf structure 133. In some examples, the sheaf structure 133 is a cellular sheaf structure. A sheaf structure 133 is integrated with the graph 110 (or portion thereof). In some examples, the sheaf structure 133 is disposed over the graph 110. A sheaf structure 133 over a graph 110 may be an object associating a space with each node 106 and edge 108 in the graph 110 and a map between these spaces for each incident node-edge pair.

The computing system 100 may obtain a graph representation of at least a portion of the graph 110 and input the graph representation to the GNN model 101. In some examples, the graph representation includes the feature coordinates 114 (e.g., features of the nodes 106). In some examples, the graph representation includes the feature coordinates 114 and the positional coordinates 116 (e.g., positions of the nodes 106). In some examples, the feature coordinates 114 and the positional coordinates 116 are arranged in a matrix (e.g., an adjacency matrix). In some examples, the graph representation includes the input embedding 112. In some examples, the graph representation does not include an embedding. In some examples, the graph representation includes the sheaf structure 133.

Referring to FIG. 1B, at least a portion of the graph 110 is input into an input layer 120(1) in the GNN model 101. The GNN model 101 includes a plurality of layers 120(1), 120(2), . . . , 120(N), where layer 120(N) is an output layer. The output layer 120(N) generates an output 130. In some examples, the output 130 includes at least a portion of a modified graph 110a (e.g., modified by the GNN model 101). In some examples, the output 130 includes feature coordinates 114a representing a second set of features for at least a portion of the nodes 106 of the graph 110. The second set of features associated with a respective node 106 may be an updated version (or evolved version) of the first set of features, where updates (or evolution) are implemented by one or more diffusion processes 121 (as discussed later in the disclosure). In some examples, the output 130 includes positional coordinates 116a representing a second position of each of at least a portion of the nodes 106. The second position associated with a respective node 106 may be an updated version (or evolved) of the first position, where the updates are implemented by one or more diffusion processes 121. In some examples, the output 130 includes an output embedding 112a, where the output embedding 112a includes the feature coordinates 114a and the positional coordinates 116a. In some examples, the output 130 includes a node embedding (e.g., the feature coordinates 114a), an edge embedding (e.g., the positional coordinates 116a). In some examples, the output embedding 112 may be input into a learning loss function 125 that is optimized to determine, e.g., node labels. In some examples, the node labels may indicate whether the user is a malicious actor.

Each layer, e.g., layer 120(2), includes a skip connection 122, a sampling operator 124, a convolution or recurrence operator 126, and a pooling operator 128. In some implementations, the pooling operator 128 is not used. The sampling operator 124 may be combined with the convolution/recurrence operator 126 to form a propagation module configured to propagate information between nodes 106. The skip connection 122 may be configured to gather previous information from nodes 106. The pooling operator 128 may be configured to extract current information from nodes 106.

The plurality of layers 120(1), 120(2), . . . , 120(N) is defined by a discretization scheme 140 for solving an underlying continuous diffusion equation (e.g., diffusion equation 127). In some examples, the continuous diffusion equation includes spatial derivatives. In some examples, the spatial derivatives include Laplace-Beltrami operators. A detailed discussion of Laplace-Beltrami operators is provided below herein.

The GNN model 101 is configured to apply one or more diffusion processes 121 to the graph representation of at least a portion of the graph 110. In some examples, the GNN model 101 applies a diffusion process 121-1 to evolve (e.g., update) the feature coordinates 114 of each node 106 in the graph representation. The evolution of feature coordinates 114 represents message passing layers (e.g., 120(1)-120(N) in the GNN model 101. In other words, the feature coordinates 114 representing the first set of features of each node 106 in the graph representation is updated by the diffusion process 121-1 to the feature coordinates 114a representing the second set of features of each node 106 in the graph representation.

In some examples, the diffusion process 121-1 is defined by a set of parameters 123. The parameters 123 may define the discretization scheme 140 for the underlying diffusion (flow) equations that dictate the evolution of nodal positions (e.g., the positional coordinates 116) and features (e.g., feature coordinates) in the graph 110. In some examples, the parameters 123 are learned by the GNN model 101 using a learning loss function 125. In some examples, the parameters 123 are learned to minimize the learning loss function 125.

In some examples, the diffusion process 121-1 includes sheaf diffusion 129. In some examples, sheaf diffusion 129 is a spatially discretized sheaf diffusion process defined by a partial differential equation (e.g., differential equation 127). In some examples, the sheaf structure 133 is learnable. In some examples, the sheaf structure 133 is learnable by the learning loss function 125. In some examples, the advantage of learning a sheaf structure 133 is that the computing system 100 may not require embeddings of the nodes 106 in ambient space. Instead, information about the sheaf structure 133 can be learned locally.

In some examples, the diffusion process 121-1 includes or is described by a differential equation 127. In some examples, the differential equation 127 includes a partial differential equation. In some examples, the diffusion process 121-1 or the differential equation 127 includes or is defined by a diffusion kernel 113. In some examples, the diffusion kernel 113 may represent the differential equation 127 from which the discretization (e.g., the discretization scheme) produces the discrete operators (e.g., Laplace-Beltrami operators) that evolve (e.g., update) the input embedding 112 to the output embedding 112a (or more generally from the first set of features to the second set of features or from the features coordinates 114 to the feature coordinates 114a). In some examples, the diffusion kernel 113 includes an attention function. In some examples, the differential equation 127 is discretized using a numerical scheme.

In some examples, the GNN model 101 applies the diffusion process 121-1 to evolve (e.g., update) the positional coordinates 116 of each node 106 in the graph representation. In other words, the positional coordinates 116 of each node 106 in the graph representation is updated by the diffusion process 121 to the positional coordinates 116a of each node 106 in the graph representation. In some examples, the position of some of the nodes 106 may change, which may indicate a rewiring of the graph 110. In some examples, the evolution of the positional coordinates 116 may represent a graph rewiring operation 131.

In some examples, the GNN model 101 defines multiple diffusion processes 121 such as the diffusion process 121-1 and diffusion process 121-2. In some examples, the diffusion process 121-1 is a node diffusion process configured to evolve (e.g., update) the first set of features (e.g., the feature coordinates 114) to the second set of features (e.g., the feature coordinates 114a). In some examples, the diffusion process 121-2 is a graph diffusion process. In some examples, the graph diffusion process is coupled to the node diffusion process. The graph diffusion process is configured to evolve the graph 110. In some examples, the graph diffusion process is based on the discrete curvature of the graph 110. In some examples, the graph diffusion process is applied first followed by the node diffusion process.

In some examples, the GNN model 101 has an architecture that is based on discretization scheme 140 for solving a continuous differential equation (e.g., differential equation 127) governing a behavior of the set of feature coordinates 114 and/or the set of positional coordinates 116 over space and time.

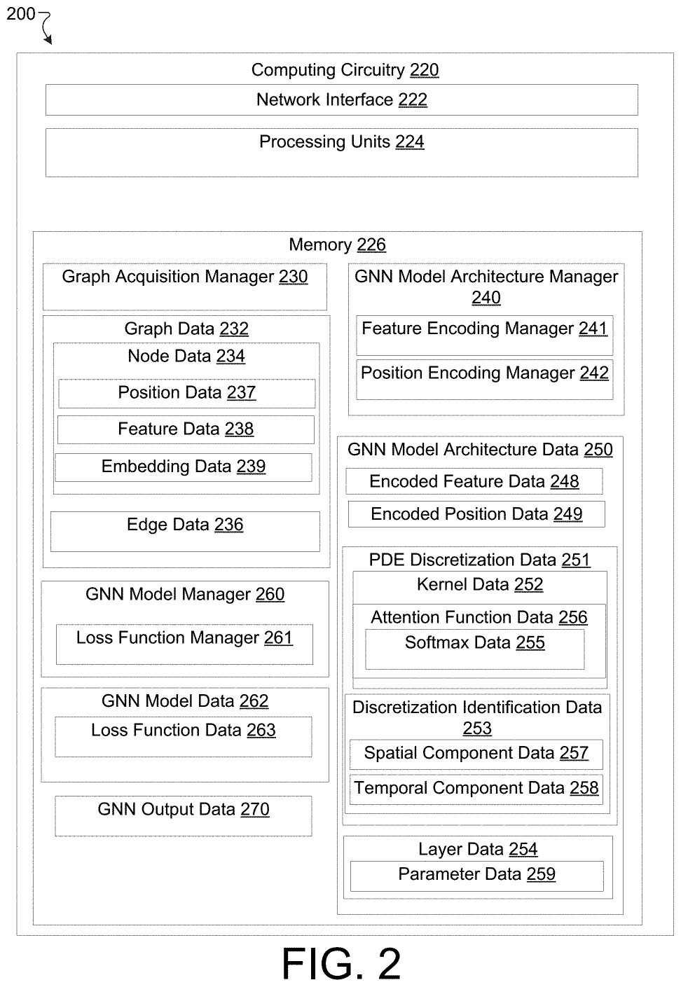

FIG. 2 is a diagram that illustrates an example electronic environment 200 in which the above-described improved techniques may be implemented. As shown, in FIG. 2, the example electronic environment 200 includes a computing circuitry 220. The computing circuitry 220 may be an example of the computing system 100 of FIGS. 1A and 1B and may include any of the details discussed with reference to those figures. Although some parts of the description are explained with reference to a messaging system, the example electronic environment 200 may be applied to any system involving a GNN model.

The computing circuitry 220 is configured to generate and/or execute a GNN model (e.g., GNN model 101 of FIGS. 1A and 1B) based on a discretization scheme (e.g., discretization scheme 140 of FIG. 1) of an underlying diffusion equation (e.g., differential equation 127 of FIG. 1A) having a diffusion kernel (e.g., diffusion kernel 113 of FIG. 1A) being based on an optimization of an action functional with respect to the embedding vector (e.g., input embedding 112 of FIG. 1A) and a metric defined by the metric space. The computing circuitry 220 includes a network interface 222, one or more processing units 224, and memory 226. The network interface 222 includes, for example, Ethernet adaptors, Token Ring adaptors, and the like, for converting electronic and/or optical signals received from a network (not shown) to electronic form for use by the computing circuitry 220. The set of processing units 224 include one or more processing chips and/or assemblies. The memory 226 includes both volatile memory (e.g., RAM) and non-volatile memory, such as one or more ROMs, disk drives, solid state drives, and the like. The set of processing units 224 and the memory 226 together form control circuitry, which is configured and arranged to carry out various methods and functions as described herein.

In some embodiments, one or more of the components of the computing circuitry 220 can be, or can include processors (e.g., processing units 224) configured to process instructions stored in the memory 226. Examples of such instructions as depicted in FIG. 2 include a graph acquisition manager 230, a GNN model architecture manager 240, and a GNN model manager 260. Further, as illustrated in FIG. 2, the memory 226 is configured to store various data, which is described with respect to the respective managers that use such data.

The graph acquisition manager 230 is configured to acquire graph data 232 (e.g., at least a portion of graph 110 of FIGS. 1A and 1B) for input into a GNN Model (e.g., GNN model 101 of FIGS. 1A and 1B) for, e.g., classification of nodes. In some implementations, the graph acquisition manager 230 is configured to receive graph data 232 over the network interface 222 via a network (not shown). In some implementations, the graph acquisition manager 230 is configured to receive graph data 232 via a local storage device, e.g., a flash drive, a disk drive, a RAID system, or the like. In some implementations, the graph acquisition manager 230 is configured to generate graph data 232 based on, e.g., input from a social network or the like.

The graph data 232 represents an input graph (e.g., input graph 110 of FIGS. 1A and 1B). In some implementations, the graph data 232 is arranged as a list of nodes (vertices) and edges. For example, as shown in FIG. 2, the graph data 232 includes node data 234 and edge data 236. In some implementations, the graph data 232 is arranged as a matrix, e.g., an adjacency matrix. For example, the graph data 232 may represent a network of connected users in a social media message system that recommends information to the users.

The node data 234 represents the nodes in the graph represented by the graph data 232. The topology of the graph, i.e., the placement of the nodes within the graph may be deduced, in some implementations, from the edge data 236. In some implementations, the node data 234 includes coordinates in a coordinate system defined within the graph. As shown in FIG. 2, the node data 234 includes feature data 238 and position data 237.

The position data 237 (e.g., U in Eq. (8)) (Eq. (8) is further described below) represents vertex positions within the graph. In some implementations, the position data 237 is expressed in a Euclidean coordinate system. In some implementations, the position data 237 is expressed in a non-Euclidean coordinate system with metric d_{C}. In some implementations, the non-Euclidean coordinate system is a hyperbolic coordinate system.

The feature data 238 (e.g., X in Eq. (8)) represents features associated with their nodes. In an example, in a social network application, the node data 234 may represent users within the network. In that case, the feature data 238 may represent additional information about each user (e.g., age, occupation, hometown, etc.). In some implementations, the feature data 238 represents posts (e.g., messages) created by the users.

In some implementations, the feature data 238 and position data 237 are combined (e.g., concatenated) to form an initial embedding vector, i.e., embedding data 239 (e.g., Z in Eq. (10)) (Eq. (10) is further described below). In some implementations, the initial embedding vector is defined in a metric space having an input metric g_{M}. In some implementations, the input metric is non-Euclidean. In some implementations, the metric evolves with time, i.e., flows, e.g., according to Eq. (46) (Eq. (46) is further described below).

The edge data 236 represents edges in the graph, i.e., connections or relationships between pairs of nodes. For example, if the graph represents a social network, the edges can represent a “friend” relationship between users represented by the nodes. In some implementations, the edge data 236 is expressed in terms of the nodes linked by each edge. In some implementations, the edge data 236 also includes information about directionality, i.e., when the graph is directional. In some implementations, the edge data 236 is represented as pairs of nodes. In some implementations, the order of the nodes listed in each pair indicate a direction of that edge. In some implementations, the edge data 236 includes edge features, which may include information about each edge, e.g., an edge classification.

The GNN model architecture manager 240 is configured to generate a GNN model architecture based on a discretization of an underlying diffusion, represented in the GNN Model Architecture Data 250. The GNN model architecture manager 240 is defined by a set of layers (e.g., GNN layers 120(1) to 120(N) of FIG. 1B), each of which include parameter values that may or may not change with time and hence be shared across layers. As shown in FIG. 2, the GNN model architecture manager 240 includes a feature encoding manager 241 and a position encoding manager 242.

The feature encoding manager 241 is configured to encode the feature data 238 according to an encoding function. In some implementations, the encoding function is learnable, e.g., encoding function ψ(Xᵢₙ).

The position encoding manager 242 is configured to encode the position data 237 according to an encoding function. In some implementations, the encoding function is learnable, e.g., encoding function ϕ(Uᵢₙ).

The GNN Model Architecture Data 250 represents the GNN model architecture. As shown in FIG. 2, the GNN Model Architecture Data 250 includes encoded feature data 248 (e.g., ψ(Xᵢₙ), encoded position data 249 (e.g., ϕ(Uᵢₙ)), PDE discretization data 251, and layer data 254.

The PDE discretization data 251 represents parameter values (e.g., the parameters 123 of FIG. 1A) defining the discretization scheme for the underlying diffusion (flow) equations that dictate the evolution of nodal positions and features in a graph. As shown in FIG. 2, the PDE Discretization Data 251 includes kernel data 252 and Discretization Identification Data 253.

The Kernel Data 252 represents the underlying diffusion equation from which the discretization produces the discrete operators that evolve (e.g., update) the input embedding to produce the final embedding. As shown in FIG. 2, the kernel data 252 includes attention function data 256.

The attention function data 256 represents the attention function, e.g., the matrix α(zᵢ^{(k)}, zⱼ^{(k)}) in Eq. (9) or the matrix Q^{(k)} in Eq. (10) (Eq. (10) is further described below). The mathematical form of the attention function may depend on the spatial discretization scheme identified in the Discretization Identification Data 253. The attention function is, in some implementations, learnable; to this effect, the attention function data 256 includes softmax data 255. The softmax data 255 represents at least one learnable matrix and at least one hyperparameter (e.g., Eq. (13) (Eq. (13) is further described below)).

The Discretization Identification Data 253 represents the discretization scheme for solving the underlying diffusion equation (e.g., diffusion equation 127 of FIG. 1A) governing the flow of information through the vertices of the graph. As shown in FIG. 2, the Discretization Identification Data 253 includes Spatial Component Data 257 and Temporal Component Data 258.

The Temporal Component Data 258 represents the type of time difference used in the approximation of the time derivative in Eq. (7) (Eq. (7) is further described below). For example, a temporal discretization scheme is the forward time difference used in Eq. (9) (Eq. (9) is further described below); this case, the Temporal Component Data 258 includes an identifier identifying the temporal scheme as forward difference and a time step value. In some implementations, the temporal discretization scheme is a RK scheme of order at least four. In some implementations, the temporal discretization scheme is a Dormand-Prince scheme. The scheme and time step value, in some implementations, determines the layer architecture, e.g., number of hidden layers.

The Spatial Component Data 257 represents a scheme to approximate the gradients to produce the elements of the attention function (attention function data 256). In some implementations, the Spatial Component Data 257 identifies the scheme as a Moving Mesh (MM) method.

The Layer Data 254 represents the layers of the GNN—the initial, hidden, and final layers—in which the positional and feature coordinates evolve from initial to final states. As shown in FIG. 2, the layer data 254 includes parameter data 259 representing the values of the learned parameters defining the evolution of the positional and feature coordinates. In some implementations, when the attention matrix is time-independent, the values of the learned parameters are shared between the layers.

The GNN Model Manager 260 is configured to train a model to generate GNN Model Data 262, i.e., the various learnable functions/parameters used in the GNN model, e.g., for the attention function, the matrices W_{K} and W_{Q} in Eq. (13) (Eq. (13) is further described below), according to a loss function (e.g., the learning loss function 125) defined by Loss Function Manager 261 included in the GNN Model Manager 260.

The GNN Model Data 262 represents the GNN model used to train the various learnable functions/parameters used in the GNN model. As shown in FIG. 2, the GNN Model Data 262 includes Loss Function Data 263, which represents the loss function, i.e., parameters defining a particular loss function.

The GNN Output Data 270 represents the final embedding (i.e., positional and feature coordinates) as generated by the GNN model, e.g., Y=ξZ(T)) in BLEND.

In some implementations, the graph data 232 represents an image for input into an image classifier or an edge-preserving image denoiser. In some implementations, the graph data 232 represents a system of molecules used in drug discovery; in such implementations, the GNN model is used to classify molecules that are likely or unlikely to become a useful drug.

The components (e.g., modules, processing units 224) of the computing circuitry 220 can be configured to operate based on one or more platforms (e.g., one or more similar or different platforms) that can include one or more types of hardware, software, firmware, operating systems, runtime libraries, and/or so forth. In some implementations, the components of the computing circuitry 220 can be configured to operate within a cluster of devices (e.g., a server farm). In such an implementation, the functionality and processing of the components of the computing circuitry 220 can be distributed to several devices of the cluster of devices.

The components of the computing circuitry 220 can be, or can include, any type of hardware and/or software configured to process attributes. In some implementations, one or more portions of the components shown in the components of the computing circuitry 220 in FIG. 2 can be, or can include, a hardware-based module (e.g., a digital signal processor (DSP), a field programmable gate array (FPGA), a memory), a firmware module, and/or a software-based module (e.g., a module of computer code, a set of computer-readable instructions that can be executed at a computer). For example, in some implementations, one or more portions of the components of the computing circuitry 220 can be, or can include, a software module configured for execution by at least one processor (not shown). In some implementations, the functionality of the components can be included in different modules and/or different components than those shown in FIG. 2, including combining functionality illustrated as two components into a single component.

Although not shown, in some implementations, the components of the computing circuitry 220 (or portions thereof) can be configured to operate within, for example, a data center (e.g., a cloud computing environment), a computer system, one or more server/host devices, and/or so forth. In some implementations, the components of the computing circuitry 220 (or portions thereof) can be configured to operate within a network. Thus, the components of the computing circuitry 220 (or portions thereof) can be configured to function within various types of network environments that can include one or more devices and/or one or more server devices. For example, the network can be, or can include, a local area network (LAN), a wide area network (WAN), and/or so forth. The network can be, or can include, a wireless network and/or wireless network implemented using, for example, gateway devices, bridges, switches, and/or so forth. The network can include one or more segments and/or can have portions based on various protocols such as Internet Protocol (IP) and/or a proprietary protocol. The network can include at least a portion of the Internet.

In some implementations, one or more of the components of the computing circuitry 220 can be, or can include, processors configured to process instructions stored in a memory. For example, a graph acquisition manager 230 (and/or a portion thereof), a GNN model architecture manager 240 (and/or a portion thereof), and a GNN model manager 260 (and/or a portion thereof) can be a combination of a processor and a memory configured to execute instructions related to a process to implement one or more functions.

In some implementations, the memory 226 can be any type of memory such as a random-access memory, a disk drive memory, flash memory, and/or so forth. In some implementations, the memory 226 can be implemented as more than one memory component (e.g., more than one RAM component or disk drive memory) associated with the components of the computing circuitry 220. In some implementations, the memory 226 can be a database memory. In some implementations, the memory 226 can be, or can include, a non-local memory. For example, the memory 226 can be, or can include, a memory shared by multiple devices (not shown). In some implementations, the memory 226 can be associated with a server device (not shown) within a network and configured to serve the components of the computing circuitry 220. As illustrated in FIG. 2, the memory 226 is configured to store various data, including graph data 232, GNN model architecture data 250, GNN model data 262, and GNN output data 270.

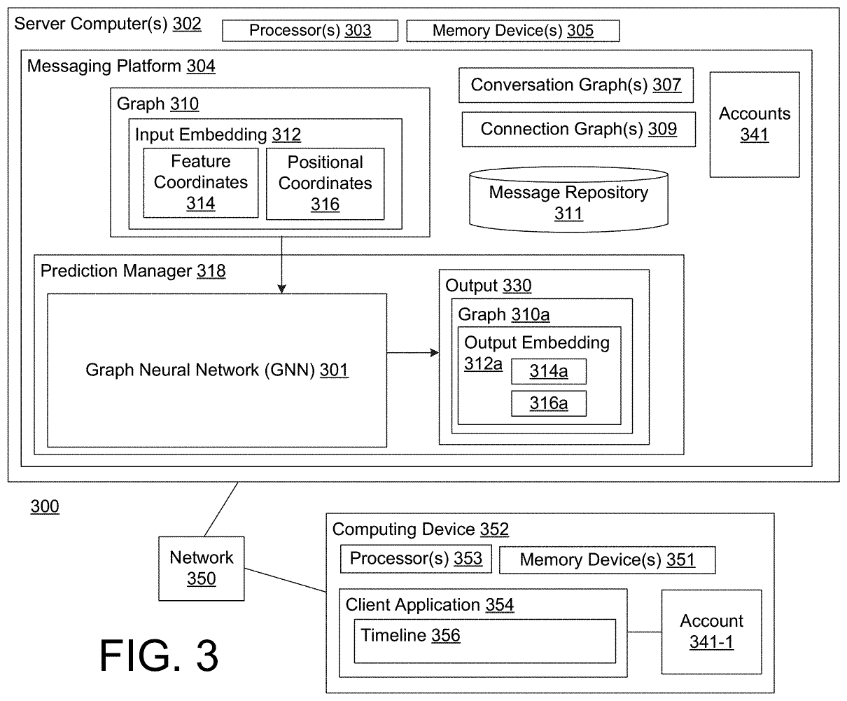

FIG. 3 illustrates a messaging system 300 according to an aspect. The messaging system 300 is configured to facilitate the exchange of messages among users of the messaging system 300. The messaging system 300 includes a messaging platform 304 executable by one or more server computers 302, and a client application 354 executable by a computing device 352 according to an aspect. The client application 354 communicates with the messaging platform 304 to send (and receive) messages, over a network 350, to (and from) other users (e.g., accounts 341) of the messaging platform 304.

The messaging platform 304 includes a prediction manager 318. The prediction manager 318 may be an example of the computing system 100 of FIGS. 1A and 1B and/or the computing circuitry 220 of FIG. 2 and may include any of the details with respect to those figures.

The prediction manager 318 may use the GNN model 301 to recommend information to users. In some examples, the prediction manager 318 may use the GNN model 301 to recommend messages, topics, interests, user accounts 341 to establish a connection (e.g., follow, friend, etc.) in a connection graph 307, news articles, and/or promoted information such as promoted messages or profiles. In some examples, the prediction manager 318 may use the GNN model 301 to classify information posted on the messaging platform 304 (e.g., messages, hashtags, terms used within messages, etc.), information determined or detected by the messaging platform 304, and/or classify users of the messaging platform 304 such as labeling user accounts 341 that violate one or more terms of the messaging platform 304 (e.g., malicious actors posting toxic, abusive, an/or spam messages). In some examples, the prediction manager 318 may use the GNN model 301 to cluster information posted to the messaging platform 304 and/or users of the messaging platform 304.

The client application 354 may be a social media messaging application in which users post and interact with messages. In some examples, the client application 354 is a native application executing on an operating system of the computing device 352 or may be a web-based application executing on the server computer(s) 302 (or other server) in conjunction with a browser-based application of the computing device 352. The computing device 352 may access the messaging platform 304 via the network 350 using any type of network connections and/or application programming interfaces (APIs) in a manner that permits the client application 354 and the messaging platform 304 to communicate with each other.

The computing device 352 may be a mobile computing device (e.g., a smart phone, a PDA, a tablet, or a laptop computer) or a non-mobile computing device (e.g., a desktop computing device). The computing device 352 includes one or more processors 353 and one or more memory devices 351. The processor(s) 353 may be formed in a substrate configured to execute one or more machine executable instructions or pieces of software, firmware, or a combination thereof. The processor(s) 353 can be semiconductor-based—that is, the processors can include semiconductor material that can perform digital logic. The memory device(s) 351 may include a main memory that stores information in a format that can be read and/or executed by the processor(s) 353. The computing device 352 also includes various network interface circuitry, such as for example, a mobile network interface through which the computing device 352 can communicate with a cellular network, a Wi-Fi network interface with which the computing device 352 can communicate with a Wi-Fi base station, a Bluetooth network interface with which the computing device 352 can communicate with other Bluetooth devices, and/or an Ethernet connection or other wired connection that enables the computing device 352 to access the network 350.

The server computer(s) 302 may be a single computing device or may be a representation of two or more distributed computing devices communicatively connected to share workload and resources. The server computer 302 may include at least one processor 303 and a memory device 305 that stores executable instructions that when executed by the at least one processor 303 cause the at least one processor 303 to perform the operations discussed herein.

The messaging platform 304 is a computing platform for facilitating communication (e.g., real-time communication) between user devices (one of which is shown as computing device 352). The messaging platform 304 may store millions of accounts 341 of individuals, businesses, and/or entities (e.g., pseudonym accounts, novelty accounts, etc.). One or more users of each account 341 may use the messaging platform 304 to send messages to other accounts 341 inside and/or outside of the messaging platform 304. In some examples, the messaging platform 304 may enable users to communicate in “real-time”, e.g., to converse with other users with minimal delay and to conduct a conversation with one or more other users during simultaneous sessions. In other words, the messaging platform 304 may allow a user to broadcast messages and may display the messages to one or more other users within a reasonable time frame (e.g., less than two seconds) to facilitate a live conversation between users. In some examples, recipients of a message may have a predefined graph relationship in a connection graph 309 with an account (e.g., account 341-1) of the user broadcasting the message.

The connection graph 309 includes a data structure that indicates which accounts 341 in the messaging platform 304 are associated with (e.g., following, friends with, subscribed to, etc.) a particular account 341 and are, therefore, subscribed to receive messages from the particular account 341. For example, the connection graph 309 may link a first account with a second account, which indicates that the first account is in a relationship with the second account. The user of the second account may view messages posted on the messaging platform 304 by the user of the first account (and/or vice versa). The relationships may include unidirectional (e.g., follower/followee) and/or bidirectional (e.g., friendship). The messages can be any of a variety of lengths which may be limited by a specific messaging system or protocol.

In some examples, users interested in viewing messages authored by a particular user can choose to follow the particular user. A first user can follow a second user by identifying the second user as a user the first user would like to follow. After the first user has indicated that they would like to follow the second user, the connection graph 309 is updated to reflect the relationship, and the first user will be provided with messages authored by the second user. Users can choose to follow multiple users. Users can also respond to messages and thereby have conversations with one another. In addition, users may engage with messages such as sharing a message with their followers or favoritizing (or “liking”) a message in which the engagement is shared with their followers.

The messaging platform 304 may send digital information, over the network 350, to enable the client application 354 to render and display a timeline 356 of social content on the user interface of the client application 354. The timeline 356 includes a stream of messages (e.g., message A, message B, message C). In some examples, the stream of messages are arranged in reverse chronological order. In some examples, the stream of messages are arranged in chronological order. In some examples, the timeline 356 is a timeline of social content specific to a particular user. In some examples, the timeline 356 includes a stream of messages curated (e.g., generated and assembled) by the messaging platform 304. In some examples, the timeline 356 includes a list of messages that resulted from a search on the messaging platform 304. In some examples, the timeline 356 includes a stream of messages posted by users from accounts 341 that are in relationships with the account 341 of the user account 341-1 of the client application 354 (e.g., a stream of messages from accounts 341 that the user account 341-1 has chosen to follow on the messaging platform 304). In some examples, the stream of messages includes promoted messages or messages that have been re-shared.

Messages exchanged on the messaging platform 304 are stored in message repository 311. The message repository 311 may include one or more tables storing records. In some examples, each record corresponds to a separately stored message. For example, a record may identify a message identifier for the message posted to the messaging platform 304, an author identifier (e.g., @tristan) that identifies the author of the message, message content (e.g., text, image, video, and/or URL of web content), one or more participant account identifiers that have been identified in the body of the message, and/or reply information that identifies the parent message for which the message replies to (if the message is a reply to a message).

The messaging platform 304 may include one or more conversation graphs 307. In some examples, the conversation graphs 307 are stored in a data storage device associated with the messaging platform 304. The messaging platform 304 may store multiple conversation graphs 307 (e.g., hundreds, thousands, or millions of conversation graphs 307). Each conversation graph 307 may represent a structure of replies to an original, non-reply message (e.g., a root message). For example, whenever a user creates and posts an original, non-reply message on the messaging platform 304, a potential new conversation may be started. Others can then reply to that original or “root” message and create their own reply branches. Over time, if the number of replies to the original, non-reply message (and/or replies to the replies to the original, non-reply message) is greater than a threshold level, the messaging platform 304 may assign a conversation identifier to the conversation graph 307. The conversation graph 307 may be a hierarchical data structure representing the messages in a conversation. In some examples, the conversation graph 307 includes a nonlinear or linear data structure. In some examples, the conversation graph 307 includes a tree data structure.

The GNN model 301 is configured to receive at least a portion of a graph 310. In some examples, the graph 310 is the connection graph 309 (or a portion thereof). In some examples, the graph 310 is a conversation graph 307 (or a portion thereof). The graph 310 may include a plurality of nodes (e.g., nodes 106 of FIG. 1A) and a plurality of edges (e.g., edges 108 of FIG. 1B). The nodes may represent the user accounts 341. For example, each node may correspond to a separate user account 341 of the messaging platform 304. The edges may represent the connections between the user accounts 341 of the messaging platform 304. An edge may connect a pair of nodes. For example, a first user account enters into a relationship (e.g., follows, friends, etc.) with a second user account, an edge is generated between the first user account and the second user account.

In some examples, the graph 310 includes an input embedding 312 associated with each node of the graph 310. For example, the input embedding vector 312 may include feature coordinates 314 associated with a respective user account 341 and positional coordinates 316 of a respective node within the graph 310. The feature coordinates 316 may include information about the respective user account 341 and/or information about the respective user account's behavior on the messaging platform 304 (e.g., which messages were posted, which messages were favoritized or re-shared, etc.). The positional coordinates 316 may identify a location of a respective node in the context of the overall graph 310.

The GNN model 301 may receive the input embedding 312 for the nodes of the graph 310 and generate an output 330. The output 330 may include a modified graph 310a. In some examples, the modified graph 310a may be referred to as a rewired graph, where the edges have changed. In some examples, the output 330 includes an output embedding 312a for each of the nodes of the graph 310. The output embedding 312a may include feature coordinates 314a and positional coordinates 316a, but, in some examples, the position for one or more nodes may have changed, therefore being a rewired graph 310a that can reduce the graph bottlenecks and/or reduce the distortion of information from distant nodes (thereby improving the prediction efficiency of the GNN model 301).

In some implementations, the output 130 includes updates to the features represented by the feature coordinates 314 (e.g., feature vectors) of nodes from other distant nodes. That is, due to bottlenecking some feature updates may not arrive at a node. For example, in a social network a first user represented by a first node undergoes some change (e.g., is designated as a malicious actor) that changes the feature vector describing the first user. Such an update may propagate to other nodes along edges according to, e.g., the diffusion kernel 113 of FIGS. 1A and 1B. The diffusion kernel 113 is so defined such that the updates arrive at a second node from the first node (e.g., second user receives indication that the first user has been designated as a malicious actor), even when the second node is distant from the first node.

In some implementations, the output 130 includes a graph 110a having a new set of edges resulting from a rewiring operation (e.g., the rewiring operation 131 of FIG. 1A). In some implementations, the rewiring operation is a result of the diffusion kernel 113 acting on the node positions (e.g., the positional coordinates 116). This rewiring is advantageous over conventional rewiring because the rewiring operation described herein is applied automatically as a result of performing a diffusion process (e.g., diffusion process 121 of FIG. 1A) within the GNN model 301 rather than as a separate process from updating the features (e.g., the feature coordinates 114). This automatic application of the rewiring operation results in a more efficient process than that for conventional rewiring.

In some implementations, the prediction manager 318 that may use the output 330 of the GNN model 301 to identify one or more user accounts 341 predicted to violate one or more terms of the messaging platform 304. In some examples, the identified user accounts 341 may be accounts that are identified as potential malicious actors. In some examples, the prediction manager 318 may use the output 330 of the GNN model 301 to identify one or more user accounts 341 that would be recommendations for a particular user account 341. In some implementations, such an identification is made via the diffusion kernel 113 of the GNN model 101 of FIG. 1A.

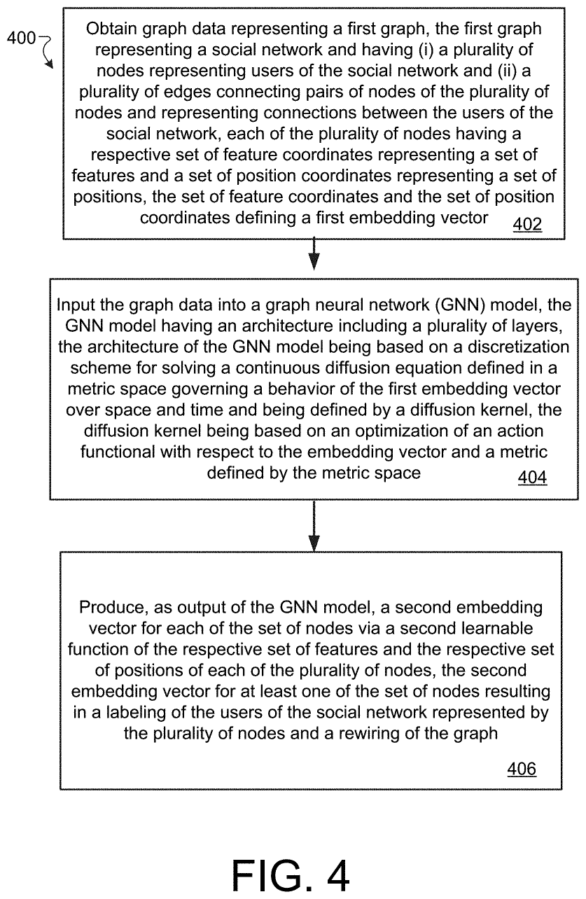

FIG. 4 illustrates a flow chart illustrating an example method 400 of performing an implementation of the improved techniques. The method 400 may be performed by software constructs described in connection with FIG. 2, which reside in memory 226 of the computing circuitry 220 and are run by the set of processing units 224. Although the flowchart of FIG. 4 is explained with reference to FIG. 2, the operations of FIG. 4 may be implemented by any of the systems discussed herein including the computing system 100 of FIGS. 1A and 1B and/or the messaging system 300 of FIG. 3. Although the flowchart of FIG. 4 illustrates the operations in sequential order, it will be appreciated that this is merely an example, and that additional or alternative operations may be included. Further, operations of FIG. 4 and related operations may be executed in a different order than that shown, or in a parallel or overlapping fashion.

At 402, the graph acquisition manager 230 obtains graph data representing a first graph (e.g., embedding vectors zᵢ, Eq. (7)), the first graph representing a social network and having (i) a plurality of nodes (e.g., set of nodes V) representing users of the social network and (ii) a plurality of edges (e.g., set of edges E) connecting pairs of nodes of the plurality of nodes and representing connections between the users of the social network, each of the plurality of nodes having a respective set of feature coordinates (e.g., X, Eq. (8)) representing a set of features and a set of positional coordinates (e.g., U, Eq. (8)) representing a set of positions, the set of feature coordinates and the set of positional coordinates defining a first embedding vector (e.g., embedding vectors zᵢ, Eq. (7)).

At 404, the GNN model manager 260 inputs the graph data into a graph neural network (GNN) model (e.g., GNN Model Data 262), the GNN model having an architecture (e.g., GNN Model Architecture Data 250) including a plurality of layers (e.g., layer data 254), the architecture of the GNN model being based on a discretization scheme (e.g., PDE Discretization Data 251) for solving a continuous diffusion equation defined in a metric space governing a behavior of the first embedding vector over space and time and being defined by a diffusion kernel (e.g., kernel data 252), the diffusion kernel being based on an optimization of an action functional with respect to the embedding vector and a metric defined by the metric space.

At 406, the GNN model manager 260 produces, as output of the GNN model, a second embedding vector (e.g., Y=ξ(Z(T))) for each of the set of nodes via a second learnable function (e.g., of the respective set of features and the respective set of positions of each of the plurality of nodes, the second embedding vector for each of the set of nodes resulting in a labeling of the users of the social network represented by the plurality of nodes and a rewiring of the graph.

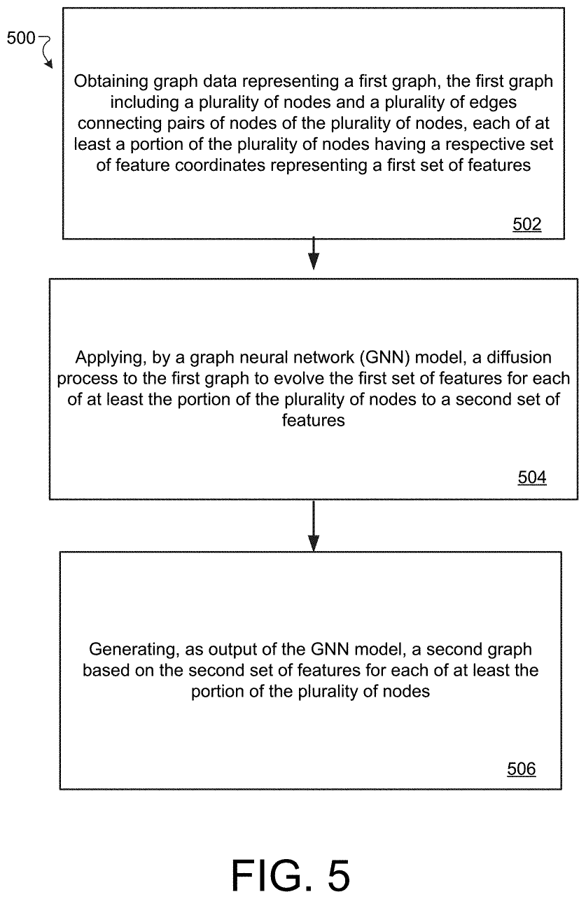

FIG. 5 illustrates a flow chart illustrating an example method 500 of performing an implementation of the improved techniques. Although the flowchart of FIG. 5 is explained with reference to the computing system 100 of FIGS. 1A and 1B, the operations of FIG. 5 may be implemented by any of the systems discussed herein including messaging system 300 of FIG. 3 and/or the software constructs described in connection with FIG. 2, which reside in memory 226 of the computing circuitry 220 and are executed by the set of processing units 224. Although the flowchart of FIG. 5 illustrates the operations in sequential order, it will be appreciated that this is merely an example, and that additional or alternative operations may be included. Further, operations of FIG. 5 and related operations may be executed in a different order than that shown, or in a parallel or overlapping fashion.

Operation 502 includes obtaining graph data representing a first graph (e.g., graph 110). The first graph includes a plurality of nodes 106 and a plurality of edges 108 connecting pairs of nodes 106 of the plurality of nodes 106. Each of at least a portion of the plurality of nodes 106 having a first set of features. Operation 504 includes applying, by a graph neural network (GNN) model 101, a diffusion process 121 to the first graph to evolve (e.g., update) the first set of features to a second set of features. Operation 506 includes generating, as output 130 of the GNN model 101, a second graph (e.g., graph 110a) based on the second set of features for each of at least the portion of the plurality of nodes 106.

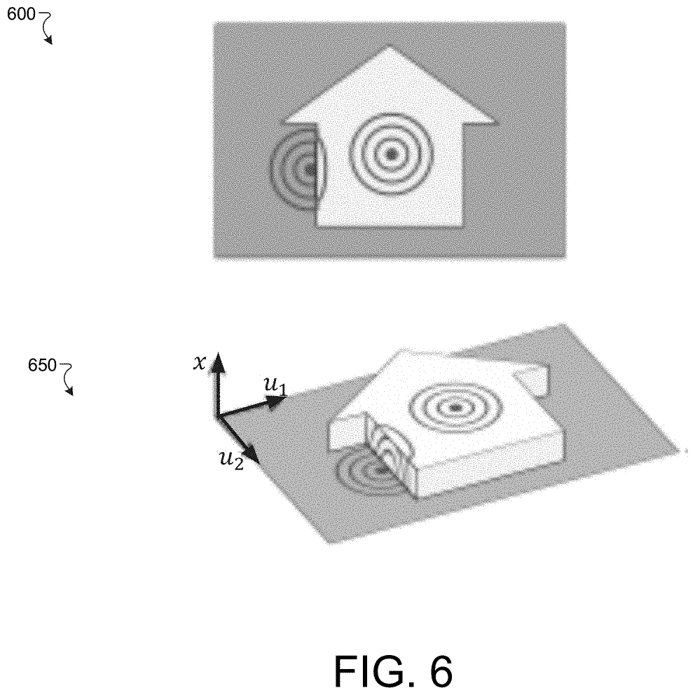

FIG. 6 is a diagram illustrating two example interpretations of Beltrami flow. The first interpretation 600 represents a position-dependent bilateral kernel. The second interpretation 650 represents a Gaussian passed on the manifold; this is represented in Eq. (5). Note that, in the case of an image, the flow averages features, e.g., color.

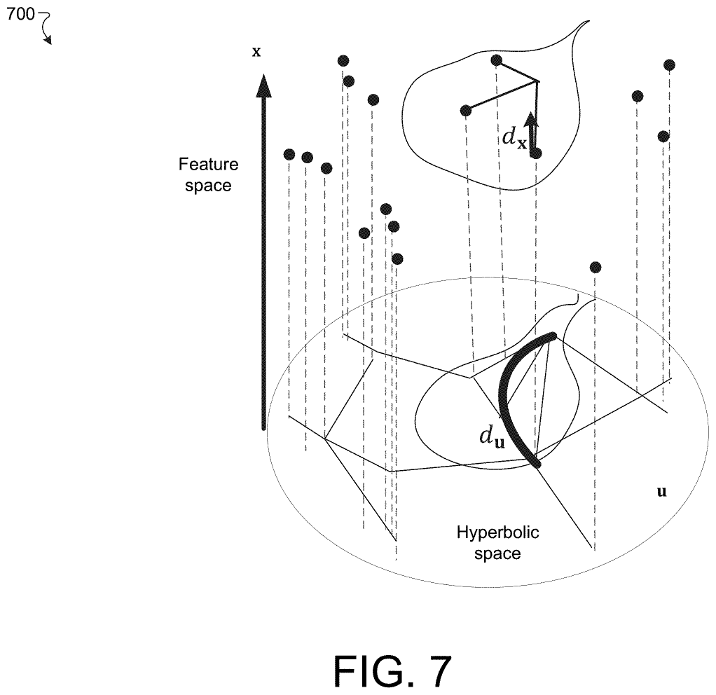

FIG. 7 is a diagram that illustrates an example Beltrami flow in a hyperbolic coordinate system 700. As shown in FIG. 7, the metric dᵤ for the positional coordinates U corresponds to hyperbolic coordinates. Hyperbolic coordinates may allow for a significant reduction in model size with only a marginal degradation in performance compared with coordinate systems using a Euclidean metric or other non-Euclidean metric. Some empirical and theoretical results indicate an advantage of using hyperbolic metric spaces to represent real-life “small-world” graphs, i.e., scale-free networks may be obtained as kNN graphs in hyperbolic spaces. There is also a metric d_{X} associated with the feature coordinates; as shown in FIG. 7, this metric is not associated with the hyperbolic space.

Referring back to FIG. 1B, the plurality of layers 120(1), 120(2), . . . , 120(N) are defined by a discretization scheme 140 for solving an underlying continuous diffusion equation in which the spatial derivatives are Laplace-Beltrami operators. A discussion of the Laplace-Beltrami operators as applied to edge-preserving denoising of images follows.

I. Graph Beltrami Flow

Images may be considered as 2-manifolds (parametric surfaces) (Σ,g) embedded in some larger ambient space as z(u)=(u, αx(u))⊆ [symbol] ^{d+2} where α>0 is a scaling factor, u=(u₁,u₂) are the 2D positional coordinates of the pixels, and x are the d-dimensional color or feature coordinates (with d=1 or 3 for grayscale or RGB images, or d=k² when using k×k patches as features). In such a consideration, the image is evolved along the gradient flow of a functional S[z, g] called the Polyakov action, which roughly measures the smoothness of the embedding. For images embedded in Euclidean space with the functional S minimized with respect to both the embedding z and the metric g, one obtains the following PDE:

$$\frac{\partial z ( u , t )}{\partial t} = {Δ}_{G} z ( u , t ) ; z ( u , 0 ) = z ( u ) ; t \geq 0 , \qquad ( 1 )$$

and boundary conditions as appropriate. Here Δ_{G} is the Laplace-Beltrami operator, the Laplacian operator induced on Σ by the Euclidean space into which the image is embedded. Namely, the embedding of the manifold allows one to pull-back the Euclidean distance structure on the image: the distance between two nearby points u and u+du is given by

$${dℓ}^{2} = d {u}^{T} G ( u ) d u = d {u}_{1}^{2} + d {u}_{2}^{2} + {α}^{2} {\sum }_{i = 1}^{d} d {x}_{i}^{2} , \qquad ( 2 )$$

where G=I+α² (∇ᵤx(u))^{T}∇ᵤx(u) is a 2×2 matrix called the Riemannian metric. The fact that the distance is a combination of the positional component (distance between pixels in the plane, ∥u−u′∥) and color component (distance between the colors of the pixels, ∥x(u)−x(u′)∥) allows edge-preserving image diffusion.

When dealing with images, the evolution of the first two components of (z₁,z₂)=u is a nuisance amounting to the reparametrization of the manifold and can be ignored. For grayscale images (the case when d=1 and z=(u₁,u₂,x)), this is done by projection along the dimension z₃, in which case the Beltrami flow takes the form of an inhomogeneous diffusion equation of x,

$$\frac{\partial x ( u , t )}{\partial t} = \frac{1}{\sqrt{det G ( u , t )}} d i v ( \frac{∇ x ( u , t )}{\sqrt{det G ( u , t )}} ) ; t \geq 0 . \qquad ( 3 )$$

The diffusivity

$$α = \frac{1}{\sqrt{det G ( u , t )}} = \frac{1}{\sqrt{1 + {α}^{2} {‖ ∇ x ‖}^{2}}} \qquad ( 4 )$$

determining the speed of diffusion at each point, can be interpreted as an edge indicator: diffusion is weak across edges where ∥∇x∥>>1. The result is an adaptive diffusion having an ability to denoise images while preserving their edges. For cases with d>1 (multiple color channels), Eq. (3) is applied to each channel separately; nevertheless,
the metric G couples the channels, which results in their gradients becoming aligned.

In the limit case α=0, Eq. (3) becomes the simple homogeneous isotropic diffusion

$$\frac{\partial }{\partial t} x = div ( ∇ x ) = Δ x ,$$

where

$$Δ = \frac{{\partial }^{2}}{\partial {{u}_{1}}^{2}} + \frac{{\partial }^{2}}{\partial {{u}_{2}}^{2}}$$

is the standard Euclidean Laplacian operator. The solution is given in closed form as the convolution of the initial image and a Gaussian kernel with time-dependent variance,

$$x ( u , t ) = x ( u , 0 ) * \frac{1}{{( 4 π t )}^{\frac{d}{2}}} {e}^{- \frac{\vert \vert u \vert {\vert}^{2}}{4 t}} \qquad ( 5 )$$

and can be considered a simple linear low-pass filtering. In the limit t→∞, the image becomes constant and equal to the average color.

Another interpretation of the Beltrami flow is passing a Gaussian on the manifold (see FIG. 3, bottom), which can locally be expressed as non-linear filtering with the bilateral kernel dependent on the joint positional and color distance (see FIG. 3, top),

$$x ( u , t ) = \int {\int }_{{ℝ}^{2}} x ( v , 0 ) {e}^{- \frac{{‖ u - v ‖}^{2}}{4 t}} {e}^{- {α}^{2} \frac{{‖ x ( u , 0 ) - x ( v , 0 ) ‖}^{2}}{4 t}} dv . \qquad ( 6 )$$

For α=0, the bilateral filter defined in Eq. (6) reduces to a simple convolution with a time-dependent Gaussian.

The analogy of Beltrami flow for graphs may now be developed. A graph is considered to be a discretization of a continuous structure (manifold). It will be shown that the evolution of the feature coordinates in time amounts to message passing layers in GNNs, whereas the evolution of the positional coordinates amounts to graph rewiring, which is used in some GNN architectures.

Let [symbol] =( [symbol] ={1, . . . , n}, ε) be an undirected graph, where [symbol] and E denote node and edge sets, respectively. It is further assumed node-wise d-dimensional features xᵢ∈ [symbol] ^{d} for i=1, . . . , n. Denote by zᵢ=(uᵢ, αxᵢ) the embedding of the graph in a joint space [symbol] × [symbol] ^{d}, where [symbol] is a d′-dimensional space with a metric [symbol] representing the node coordinates (for simplicity, we will assume [symbol] = [symbol] ^{d′} unless otherwise stated). uᵢ and xᵢ are referred to as the positional and feature coordinates of node i, respectively, and arrange them into the matrices U, X, and Z, of sizes n×d′, n×d, and n×(d+d′).

In the case of images, the Beltrami flow amounts to evolving the embedding z along div(a(z)∇z), with a a diffusivity map. Accordingly, the graph Beltrami flow is considered to be a discrete diffusion equation of the form

$$\frac{\partial {z}_{i}}{\partial t} = {\sum }_{j : ( i , j ) ∈ {ε}^{′}} a ( {z}_{i} ( t ) , {z}_{j} ( t ) ) ( {z}_{j} ( t ) - {z}_{i} ( t ) ) ; \\ {z}_{i} ( 0 ) = {z}_{i} ; i = 1 , … , n ; t \geq 0. \qquad ( 7 )$$

The definition is motivated as follows: gᵢⱼ=zⱼ−zᵢ and dᵢ=Σ_{:(i,j)∈ε′}gᵢⱼ are the discrete analogies of the gradient ∇z and the divergence div(g), both with respect to a graph ( [symbol] , ε′) that can be interpreted as the numerical stencil for the discretization of the continuous Laplace-Beltrami operator in Eq. (3). Note that ε′ can potentially be different from the input ε (referred to as ‘rewiring’). Some GNNs use ε′=ε, i.e., input graph is used for diffusion, no rewiring. Alternatively, the positional coordinates of the nodes can be used to define a new graph topology either with ε(U)={(i,j): [symbol] (uᵢ,uⱼ)<r} for some radius r>0, or using k nearest neighbors. This new rewiring can be precomputed using the input positional coordinates (i.e., ε′=ε(U(0)) or updated throughout the diffusion (i.e., ε′(t)=ε(U(t))). Therefore, Eq. (7) can be compactly rewritten as

$$\frac{\partial {z}_{i} ( t )}{\partial t} = d i v ( a ( {z}_{i} ( t ) ) ∇ {z}_{i} ( t ) ) .$$

The function a is the diffusivity controlling the diffusion strength between nodes i and j and is assumed to be normalized: Σ_{j:(i,j)∈ε′}a(zᵢ,zⱼ)=1. The dependence of the diffusivity on the embedding z matches a smooth PDE and is consistent with a form of attention mechanism. In matrix-form, we can also rewrite Eq. (7) as

$$( \frac{\partial U ( t )}{\partial t} , \frac{\partial X ( t )}{\partial t} ) = ( A ( U ( t ) , X ( t ) ) - I ) ( U ( t ) , X ( t ) ) U ( 0 ) = U ; X ( 0 ) = α X ; t \geq 0 \qquad ( 8 )$$

where we emphasize the evolution of both the positional and feature components, coupled through the matrix-valued function A,

$${a}_{i j} ( t ) = \{ \begin{matrix}a ( ( {u}_{i} ( t ) , {x}_{i} ( t ) ) , ( {u}_{j} ( t ) , {x}_{j} ( t ) ) ) & ( i , j ) ∈ ε ( U ( t ) ) \\ 0 & ( i , j ) ∉ ε ( U ( t ) )\end{matrix}$$

representing the diffusivity. The graph Beltrami flow produces an evolution of the joint positional and feature coordinates, Z(t)=(U(t),X(t)). It may be shown how the evolution of the feature coordinates X(t) results in feature diffusion or message passing on the graph, the core of GNNs. As previously noted, in the smooth case the Beltrami flow is obtained as gradient flow of an energy functional when minimized with respect to both the embedding and the metric on the surface (an image). When the embedding takes values in the Euclidean space, this leads to equations of the form Eq. (3) with no channel-mixing and an exact form of the diffusivity determined by the pull-back G of the Euclidean metric. It is tempting to investigate whether a similar conclusion can be attained here. Although in the discrete case the operation of pull-back is not well-defined, one is able to derive that the gradient flow of a modified graph Dirichlet energy gives rise to an equation of the form Eq. (7). It is noted though that the gradient flow does not recover the exact form of the diffusivity implemented herein. This is not a limitation of the theory and should be expected: by requiring the gradient flow to avoid channel-mixing and imitate the image analogy in and by inducing a discrete pull-back condition, constraints are imposed on the problem.

Theorem 1. Under structural assumptions on the diffusivity, graph Beltrami flow in Eq. (7) is the gradient flow of the discrete Polyakov functional.

Eq. (7) may be solved numerically; in the simplest case, the continuous time derivative is replaced with a forward time difference:

$$\frac{{z}_{i}^{( k + 1 )} - {z}_{i}^{( k )}}{τ} = {\sum }_{j : ( i , j ) ∈ ε ( {U}^{( k )} )} a ( {z}_{i}^{( k )} , {z}_{j}^{( k )} ) ( {z}_{j}^{( k )} - {z}_{i}^{( k )} ) . \qquad ( 9 )$$

Here k denotes the discrete time index (iteration) and τ is the time step (discretization parameter). Rewriting Eq. (9) compactly in matrix-vector form with τ=1 leads to the explicit or forward Euler scheme:

Z^{(k+1)}=(A^{(k)}−I)Z^{(k)}=Q^{(k)}Z^{(k)}, (10)

where aᵢⱼ^{(k)}=a(zᵢ^{(k)},zⱼ^{(k)}) and the matrix Q^{(k)} (diffusion operator) is given by

$${q}_{i j}^{( k )} = \{ \begin{matrix}1 - τ {\sum }_{l : ( i , l ) ∈ E} {a}_{i l}^{( k )} & i = j \\ τ {a}_{i j}^{( k )} & ( i , j ) ∈ ε ( {U}^{( k )} ) \\ 0 & otherwise\end{matrix} .$$

The solution to the diffusion equation is computed by applying the scheme in Eq. (10) multiple times in sequence, starting from some initial Z⁽⁰⁾. It is consider explicit because the update Z^{(k+1)} is done directly by the application of the diffusion operator Q^{(k)} on Z^{(k)} (as opposed to implicit schemes of the form Z^{(k)}=Q^{(k)}Z^{(k+1)} arising from backward time differences that require inversion of the diffusion operator).

Higher-order approximation of temporal derivatives amount to using intermediate fractional steps, which are then linearly combined. Runge-Kutta (RK), ubiquitously used in numerical analysis, is a classical family of explicit numerical schemes, including Euler as a particular case. The Dormand-Prince (DOPRI) is an RK method based on fifth and fourth-order approximations, the difference between which is used as an error estimate guiding the time step size.

Many numerical PDE solvers also employ adaptive spatial discretization. The choice of the stencil (mesh) for spatial derivatives is done based on the character of the solution at these points; in the simulation of phenomena such as shock waves it is often desired to use denser sampling in the respective regions of the domain, which can change in time. A class of techniques for adaptive rewiring of the spatial derivatives are known as Moving Mesh (MM) methods. Interpreting the graph E′ in Eq. (7) as the numerical stencil for the discretization of the continuous Laplace-Beltrami operator in Eq. (3), we can regard rewiring as a form of MM.

Eq. (9) has the structure of many GNN architectures of the ‘attentional’ type, where the discrete time index k corresponds to a convolutional or attentional layer of the GNN and multiple diffusion iterations amount to a deep GNN. In the diffusion formalism, the time parameter t acts as a continuous analogy of the layers, in the spirit of neural differential equations. Conventional GNNs may amount to explicit single-step (Euler) discretization schemes, whereas the continuous interpretation can exploit more efficient numerical schemes.

The graph Beltrami framework leads to a family of graph neural networks that generalize many popular architectures. For example, GAT can be obtained as a particular setting of our framework where the input graph is fixed (ε′=ε) and only the feature coordinates X are evolved. Eq. (10) in this case becomes

$${x}_{i}^{( k + 1 )} = {x}_{i}^{( k )} + τ {\sum }_{j : ( i , j ) ∈ ε} a ( {x}_{i}^{( k )} , {x}_{j}^{( k )} ) ( {x}_{j}^{( k )} - {x}_{i}^{( k )} ) \qquad ( 11 )$$

and corresponds to the update formula of GAT with a residual connection and the assumption of no non-linearity between the layers. The role of the diffusivity is played by a learnable parametric attention function, which is generally time-dependent: a(zᵢ^{(k)}, zⱼ^{(k)}, k). This results in separate attention parameters per layer k, which can be learned independently. The intentionally simplistic choice of a time-independent attention function amounts to parameter sharing across layers; this leads to a smaller model that is less likely to overfit.

In some implementations, one may decouple the input graph from the graph used for diffusion. Such rewiring can take the form of graph sampling to address scalability issues, data denoising, removal of information bottlenecks, or larger multi-hop filters. The graph construction can also be made differentiable and a task-specific rewiring can be learned. The statement that ‘diffusion improves graph learning’, leading to the eponymous paradigm (DIGL), can be understood as a form of diffusion on the graph connectivity independent of the features. In some implementations, one may use as node positional encoding the Personalized PageRank (PPR), which can be interpreted as the steady-state of a diffusion process

$${U}_{P P R} = {\sum }_{k \geq 0} ( 1 - β ) {( β {Δ}_{R W} )}^{k} = ( 1 - β ) {( I - {βΔ}_{R W} )}^{- 1} , \qquad ( 12 )$$

where Δ_{RW} is the random walk graph Laplacian and β∈(0,1) is a parameter such that 1−β represents the restart probability. The resulting positional encoding of dimension d=n can be used to rewire the graph by kNN sampling, which corresponds to using E′=E(U_{PPR}) in this framework.

Some GNN architectures can be seen as an explicit discretization of Eq. (7) with a fixed step size. In contrast, the improved techniques provide a continuous diffusion framework offers an additional advantage of employing more efficient numerical schemes with adaptive step size. Graph rewiring of the form ε′(t)=ε(U(t)) cab be interpreted as adaptive spatial discretization (MM method).

In some implementations, the metric [symbol] for the position coordinates U is non-Euclidean, i.e., non-cartesian. In some implementations, the metric [symbol] corresponds to hyperbolic coordinates. Hyperbolic coordinates may allow for a significant reduction in model size with only a marginal degradation in performance compared with coordinate systems using a Euclidean metric or other non-Euclidean metric. Some empirical and theoretical results indicate an advantage of using hyperbolic metric spaces to represent real-life “small-world” graphs, i.e., scale-free networks may be obtained as kNN graphs in hyperbolic spaces.

In some implementations, the diffusivity function a may be assumed to be time-dependent as opposed to the time-independence that had been assumed above. The time dependence may be expressed similarly as in Eq. (10), but with time-independent updates of the form Z^{(k+1)}=Q(Z^{(k)},θ)Z^{(k)} being made into a time-dependent form Z^{(k+1)}=Q(Z^{(k)},θ^{(k)})Z^{(k)}, where θ and θ^{(k)} denote shared and layer-dependent parameters, respectively.

In some implementations, the Beltrami flow is seen as amounting to a linear aggregation with nonlinear coefficients, or the ‘attentional’ flavor of GNNs. A more general message-passing flavor is possible using a generic nonlinear equation of the form

$$\frac{\partial Z ( t )}{\partial t} = Ψ ( Z ( t ) ) .$$

Beltrami Neural Diffusion (BLEND) is a novel class of graph neural network architectures derived from the graph Beltrami framework. It is assumed an input graph [symbol] =( [symbol] ,ε) has n nodes and d-dimensional node-wise features represented as a matrix Xᵢₙ. It is further assumed there is a d′-dimensional positional encoding Uᵢₙ of the graph nodes. BLEND architectures implement a learnable joint diffusion process of U and X and runs to for a time T to produce output node embeddings Y:

$$Z ( 0 ) = ( ϕ ( {U}_{i n} ) , ψ ( {X}_{i n} ) ) ; Z ( t ) = Z ( 0 ) + {\int }_{0}^{T} \frac{\partial Z ( t )}{\partial t} dt ; Y = ξ ( Z ( T ) ) ,$$

where ϕ and ψ are learnable positional and feature encoders and ξ is a learnable decoder. In some implementations, the learnable decoder changes the output dimensions. It is noted that the parameter α in Eqs. (2) and (8) is absorbed by ψ and made learnable.

$$\frac{\partial Z ( t )}{\partial t}$$

is given by the graph Beltrami flow equation, Eq. (8), where the diffusivity (attention) function a is also learnable. The choice of attention function depends on the geometry of the positional encoding and for Euclidean encodings the following scaled dot product attention performs well, so that

$$a ( {z}_{i} , {z}_{j} ) = softmax ( \frac{{( {W}_{K} {z}_{i} )}^{T} {W}_{Q} {z}_{j}}{{d}_{k}} ) \qquad ( 13 )$$

where W_{K} and W_{Q} are learned matrices and dₖ is a hyperparameter.

Further aspects of the Beltrami flow are now analyzed in terms of harmonic map flows and Dirichlet energies in non-Euclidean metrics. This will allow consideration of channel mixing in which different channels (e.g., features) may update each other; when there is no channel mixing, each channel evolves independently.

II. Graph Diffusion and Embedding Energies

Let (M, g_{M}) be a d-dimensional Riemannian manifold. For the purposes of this analysis, it suffices to think of a Riemannian metric g as a smooth map associating to each point p∈M a positive definite inner product on the tangent space TₚM. Whenever such additional structure is available, one can introduce a notion of gradient:

Definition 1. Let f:M→ [symbol] be a smooth map. The gradient off at p∈M is the vector field ∇_{gM}f satisfying df(X)|ₚ=g_{M}|ₚ(X,∇_{gM}f) for any vector field X. Given local coordinates {xⁱ} around p∈M, one may express the gradient of f as

(∇_{gM}f)|ₚⁱ=(g_{M}|ₚ)^{ij}∂f(p). (14)

The metric g_{M} also introduces a volume form dμ(g_{M}) that enables integration of functions on M: in local coordinates, dμ(g_{M})(p)=\sqrt{det(g_{M}|ₚ)}dx, with det(g_{M}|ₚ) being the determinant of g_{M}|ₚ and dx being the standard Lebesgue measure on a coordinate patch around p∈M. Given a closed manifold (M,g_{M}) and maps ψ,ϕ: M→ [symbol] , one defines an inner product on C^{∞}(M,g_{M}) by

[symbol]

ψ,ϕ [symbol] _{(M,g}_{_{M}}₎≡∫_{M}ψϕdμ(g_{M}). (15)

Similarly, given vector fields X, Y, one defines an inner product on the space of smooth vector fields by

[symbol]

X,Y [symbol] _{TM,g}_{_{M}}₎≡∫_{M}g_{M}(X,Y)dμ(g_{M}). (16)

Definition 2. Given a manifold (M, g_{M}) one defines the divergence div_{g}_{_{M}} as (minus) the adjoint of the gradient. Explicitly, div_{g}_{_{M}} satisfies the following:

[symbol]

∇_{g}_{_{M}}f,Y [symbol] _{(TM,g}_{_{M}}₎= [symbol] f,−div_{g}_{_{M}}(Y) [symbol] _{(M,g}_{_{M}}). (17)

In coordinates the divergence of Y takes the following form:

$$d i {v}_{{g}_{M}} ( Y ) ( p ) = \frac{1}{\sqrt{det ( {g}_{M} {\vert}_{p} )}} {\partial }_{i} ( \sqrt{det ( {g}_{M} {\vert}_{p} )} {Y}^{i} ) ( p ) . \qquad ( 18 )$$

By combining the gradient with its adjoint, one may introduce an operator on manifolds, generalizing the standard Laplacian in Euclidean space.

Definition 3. The Laplace-Beltrami operator is defined as Δ_{g}_{_{M}}≡−div_{g}_{_{M}}(∇_{g}_{_{M}}). In local coordinates, this may be expressed as follows.

$${Δ}_{{g}_{M}} f = - \frac{1}{\sqrt{det ( {g}_{M} )}} {\partial }_{i} ( \sqrt{det ( {g}_{M} )} {g}_{M}^{ij} {\partial }_{j} f ) . \qquad ( 19 )$$

Definition 4. The Dirichlet energy E associated with a smooth map f:(M,g_{M})→(N,h_{N}) is defined as follows.

$$E ( f , {g}_{M} , {h}_{N} ) ≡ \frac{1}{2} {\int }_{M} e ( f ) d μ ( {g}_{M} ) , \qquad ( 20 )$$

with e(f)=|df|_{gM}², where df is the Jacobian of f. In local coordinates {xⁱ} on M and {y^{α}} on N, e(f) is as follows.

e(f)(p)=(h_{N}|_{f(p)})_{α⊕}(g_{M}|ₚ)^{ij}∂ᵢf^{α}∂ⱼf^{β}. (21)

It is noted that the quantity E(f,g_{M},h_{N}) measures the smoothness of f according to the metric structures g_{M} and h_{N} defined on M and N respectively. If (N, h_{N}) is the flat Euclidean space [symbol] ^{d}, then the previous definition coincides with the classical notion of Dirichlet energy

$$E ( f , {g}_{M} ) = \frac{1}{2} {\sum }_{α = 1}^{d} {\int }_{M} {\vert {∇}_{{g}_{M}} {f}^{α} \vert}^{2} d μ ( {g}_{M} ) .$$

Stationary points of Eq. (20) are called harmonic maps. One may precisely characterize the harmonic maps from (M,g_{M}) to (N,h_{N}) by computing the first variation of the energy E along an arbitrary direction ∂ₜf as

$$d {E}_{f} ( {\partial }_{t} f ) = - {\int }_{M} {〈 {τ}_{{g}_{M}} ( f ) , {\partial }_{t} f 〉}_{{h}_{N}} d μ ( {g}_{M} ) \qquad ( 22 )$$

where the tension field (τ_{g}_{_{M}}(f))^{α}≡Δ_{g}_{_{M}}f^{α}+^{h}^{_{N}}Γ_{βγ}^{α}∂ᵢf^{β}∂ⱼf^{γ}g_{M}^{ij}. It follows that harmonic maps may be identified by the condition

(τ_{g}_{_{M}}(f))^{α}=Δ_{g}_{_{M}}f^{α}+^{h}^{_{N}}Γ_{βγ}^{α}∂ᵢf^{β}∂ⱼf^{γ}g_{M}^{ij}=0 (23)

for 1≤α≤dim(N), with {y^{α}} local coordinates on N. Since harmonic maps often represent minimal objects, one is interested in determining when a harmonic map exists given domain and target space. To this aim, one could study the smooth counterpart of the gradient descent approach by evolving an input map f₀ along the direction of (minus) the gradient of the energy E. From this idea the harmonic map flow as the geometric PDE is introduced as follows:

∂ₜf=τ_{g}_{_{M}}(f). (24)

The idea played a pivotal role in shaping much of contemporary analysis on manifolds being precursor to Ricci flow and mean curvature flow to mention a few. It turns out that a successful model in image processing can be interpreted as harmonic map flow.

The energy defined in Eq. (20) may be extended to the graph setting. Its properties and associated gradient flow are related to the analysis of graph neural ordinary differential equations (ODEs). First, classical analysis on graphs is reviewed.

Most of the operations defined above for manifolds naturally extend on graphs. Let G=(V,E) be a simple, unweighted, undirected and connected graph. One writes an edge (i,j)∈E by i˜j and let A denote the adjacency matrix encoding the connectivity information. Let dᵢ be the degree of node i. Given a signal f:V→ [symbol] , the classical graph gradient ∇f:E→ [symbol] is defined by

$$∇ f ( i , j ) ≡ \frac{f ( j )}{\sqrt{{d}_{j}}} - \frac{f ( i )}{\sqrt{{d}_{i}}} ; ( i , j ) ∈ E . \qquad ( 25 )$$

One may also define inner products in the space of signals on nodes and edges respectively: given ψ,ϕ: V→ [symbol] and X,Y:E→ [symbol] , let

$${〈 ψ , ϕ 〉}_{V} = {\sum }_{i} ψ ( i ) ϕ ( i ) ; {〈 X , Y 〉}_{E} = {\sum }_{i \sim j} {X}_{i j} {Y}_{i j} .$$

Once inner products have been defined on C⁰(V) and C⁰(E), one may introduce the notion of graph divergence div as the adjoint of the graph gradient:

[symbol]

∇f,Y [symbol] _{E}= [symbol] f,−div Y [symbol] _{V}, (26)

For all f∈C⁰(V) and Y∈C⁰(E). One may now naturally construct a self-adjoint positive definite operator by setting

$$Δ ≡ - ( \frac{1}{2} ) d i v ∇ ,$$

where the extra factor of ½ avoids counting the same edge twice. Δ is referred to as the Laplacian on the graph. Note that one may write

$$Δ f ( i ) = f ( i ) - {\sum }_{j : i \sim j} \frac{f ( i )}{\sqrt{{d}_{i} {d}_{j}}} . \qquad ( 27 )$$

In analogy with the continuum Euclidean case, one may rely on the notion of gradient to define a classical Dirichlet energy for a map f:V→ [symbol] ^{d}. If the norm is introduced

$${\vert ∇ {f}^{α} \vert}^{2} ( i ) = {\sum }_{j \sim i} {( ∇ {f}^{α} ( i , j ) )}^{2} , \qquad ( 28 )$$

then one may define the Dirichlet energy of f to be

$$E ( f ) ≡ \frac{1}{2} {\sum }_{α = 1}^{d} {\sum }_{i} {\vert ∇ {f}^{α} \vert}^{2} ( i ) = {\sum }_{i = 1}^{d} 〈 Δ {f}^{α} , {f}^{α} 〉 . \qquad ( 29 )$$

Lemma 5. ∇_{f}_{^{α}}E(f)=2Δf^{α}.
Accordingly, the gradient flow of the classical Dirichlet energy on graphs is simply the heat equation:

∂ₜf^{α}=−½Δ_{f}_{^{α}}E=−Δf^{α}.

An aim is to consider a more general and flexible notion of graph Dirichlet energy encoding additional structure on both the graph domain and the target space, similarly to Eq. (20). Accordingly, one first introduces a family of positive definite inner products playing the role of surrogate for the Riemmanian metric in the discrete setting.

Given a node i∈V, one may consider a notion of partial derivative of some signal f at i in the direction of an edge (i,j)∈E by ∇f(i,j). Therefore, one may treat ∇f(i,j) as a surrogate tangent vector of G at i and accordingly assign to each i an inner product g|ᵢ with dimension given by dᵢ. In particular, given vector fields X and Y, one may define their inner product at i as

$${〈 X , Y 〉}_{g} ( i ) ≡ {\sum }_{J \sim i , k \sim i} {( g {\vert}_{i} )}_{j k} {X}_{i j} {Y}_{i k} .$$

Thanks to this metric structure g and to the notion of derivative given by the classical graph gradient, one may now extend the differential operators defined on manifolds to graphs as well; differently from the classical case, such operators now depend on the metric g.

Definition 6. If f:V→ [symbol] , its g-gradient is defined as follows.

$${( {∇}_{g} f ) ( i , j ) ≡ {\sum }_{k \sim i} {( g \vert}_{i} )}^{j k} ∇ f ( i , k ) . \qquad ( 30 )$$

It is noted how, similar to Eq. (14), each derivative at node i is resealed by the inverse metric at node i. Similar to the smooth case in Eq. (15), one may generalize the inner products on the space of node signals by the following.

$${〈 ψ , ϕ 〉}_{( V , g )} ≡ {\sum }_{i} ψ ( i ) ϕ ( i ) {η}_{g} ( i ) , \qquad ( 31 )$$

for all ψ,ϕ:V→R. The term η_{g}:V→ [symbol] represents a measure on G induced by the metric structure g. In the classical case, this is either set to be one or η(i)=dᵢ. In analogy with the smooth case one instead defines

$${V ∋ i ↦ {η}_{g} ( i ) ≡ {( det g \vert}_{i} )}^{\frac{1}{{d}_{i}}} . \qquad ( 32 )$$

To motivate this choice, one assumes that, consistently with the smooth case, one sets η′_{g}(i)=\sqrt{det g|ᵢ}. If one multiplies a metric g on a d-dimensional manifold M by λ, then the volume form scales as

$${λ}^{\frac{d}{2}} d μ ( g ) .$$

If one rescales similarly g|ᵢ by λ for each i∈V, then

$${η}_{λ g}^{′} ( i ) = {λ}^{\frac{{d}_{i}}{2}} {η}_{g} ( i ) .$$

Therefore, a single global resealing of the metric structure induces changes on the discrete volume form that depend on the degree of each node. On the other hand,

η_{λg}(i)=λη_{g}(i),

meaning that one is forcing the graph to scale dimensionally as a surface. This may have important implications when taking the gradient of the Dirichlet energy with respect to g. Similar to Eq. (16) one may also define an inner product on vector fields on G by

$${〈 X , Y 〉}_{( E , g )} ≡ {\sum }_{i} {〈 X , Y 〉}_{g \vert i} {η}_{g} ( i ) . \qquad ( 33 )$$

The g-divergence div_{g} is introduced as minus the adjoint of the graph gradient, meaning that for all maps f:V→ [symbol] and ψ:E→

[symbol]

[symbol]

∇_{g}f,ψ [symbol] _{(E,g)}= [symbol] f,−div_{g}ψ [symbol] _{(V,g)}.

One has the following characterization for the g-divergence of a vector field: Lemma 7. For all i∈V and for all ψ:E→ [symbol]
we have

$$( {div}_{g} ψ ) ( i ) = \frac{1}{{η}_{g} ( i )} ( {div}_{G} ( {η}_{g} ψ ) ) ( i ) . \qquad ( 34 )$$

It is noted that the formulation of the g-divergence in Lemma 7 directly matches the coordinate expression in Eq. (18) with the divergence in Euclidean coordinates replaced by the classical graph divergence div. One may now introduce the self-adjoint and positive definite operator Δ_{g} defined by

Δ_{g}=−½div_{g}(∇_{g}).

Consistently with the smooth case, ∇_{g} is referred to as a graph-Beltrami operator. It is noted that when g|ᵢ is the identity map at each node i∈V, then Δ_{g}=Δ is the classical normalized Laplacian.

One now has all the ingredients to define a generalized Dirichlet energy. Suppose first one has an embedding into Euclidean space f: (V, g)→ [symbol] ^{d}. One may generalize Eq. (28) to account for the discrete metric structure g by replacing |∇f^{α}|²(i) with |∇_{g} f^{α}|_{g}²(i). Then one defines

$$E ( f , g ) ≡ \frac{1}{2} {\sum }_{α = 1}^{d} {\sum }_{i} {\vert {∇}_{g} {f}^{α} \vert}_{g}^{2} ( i ) {η}_{g} ( i ) .$$

Similarly to the classical case one derives the equivalent formulation:

$$E ( f , g ) = \frac{1}{2} {\sum }_{α = 1}^{d} {〈 {∇}_{g} {f}^{α} , {f}^{α} 〉}_{( V , g )} . \qquad ( 35 )$$

Accordingly, a generalization to Lemma 5 may be derived as follows: Lemma 8. ∇_{f}_{^{α}}E(f,g)=2 diag(η_{g})Δ_{g}f^{α}.

The volume factor form η_{g} is positive and does not affect the sign of the energy along its gradient flow—as for example in the Beltrami flow equations for images. In fact, this is what occurs in the smooth case where the tension field τ_{g}_{_{M}} in Eq. (22) does not account for the volume form dμ(g). Inspired by the harmonic map flow in Eq. (24) one may consider the system of differential equations given by

∂ₜf^{α}=−½(diag(72 _{g}))⁻¹∇_{f}_{^{α}}E(f,g), (36)

which, according to Lemma 8, may be rewritten as follows.

∂ₜf^{α}=−Δ_{g}f^{a}. (37)

This gradient flow represents a discrete counterpart to the harmonic map flow in the case of a smooth embedding of a manifold (image) (M, g_{M}) into Euclidean space—where one does not have connection terms and hence channel mixing. When each metric g|ᵢ is equal to the identity, then Eq. (37) reduces to the standard heat equation on graphs.

Remark 9. It is noted that the graph Beltrami operator can be rewritten as follows.

$${Δ}_{g} = \frac{1}{{η}_{g}} div ∘ {F}_{g} ∘ ∇ ,$$

with F_{g}:C⁰(E, [symbol] )→C⁰(E, [symbol] ) a linear map defined by (F_{g}Y)ᵢⱼ=η_{g}(i)(g|ᵢ)^{is}Y(i,s). Consequently, exactly as in the smooth case, when we embed a graph (G, g) into Euclidean space, then the only harmonic maps in the sense of stationary points of the energy E are exactly the constant maps f∈ker Δ. Since when a signal is evolved according to Eq. (37), one has

Ė(f(t),g)=½ [symbol] η_{g}⁻¹∇_{f}E,∇_{f}E [symbol] ≤0, (38)

an over-smoothing condition with the signal evolving to the only (trivial) harmonic map is derived.

The discussion to embeddings f:(V,g),→( [symbol] ^{d},h) is generalized, with h a smooth map assigning to each point p∈ [symbol] ^{d} a positive definite symmetric matrix (h|ₚ)_{αβ}. One defines

$$E ( f , g , h ) ≡ \frac{1}{2} {\sum }_{α = 1}^{d} {\sum }_{i} {η}_{g} ( i ) {( h {\vert}_{f ( i )} )}_{αβ} {〈 {∇}_{g} {f}^{α} , {∇}_{g} {f}^{β} 〉}_{g \vert i} . \qquad ( 39 )$$

As before the energy is nonnegative and has a global minimum attained, for given g and h, at the kernel of Δ.

In the case of an embedding of an image M, the variation of E with respect to the input metric vanishes when the pull-back condition, g*_{M}=f*h_{N}, in which variation of E with respect to the input metric g*_{M} vanishes, holds. The same conclusion applies to the graph setting.

Proposition 10. Given f:(V,g)→( [symbol] ^{d},h), ∇_{g}E(f,g,h)=0 if and only if for each i∈V and for each j, k such that (i,j), (i,k)∈E,

(g|ᵢ)ⱼₖ=(h|_{f(i)})_{αβ}∇f^{α}(i,j)∇f^{β}(i,k).

From now on whenever g satisfies the conditions in the theorem above the notation g* is used. When such condition is satisfied the energy takes the form

$$E ( f , {g}^{*} , h ) = \frac{1}{2} {\sum }_{i} {d}_{i} {η}_{g} ( i ) = \frac{1}{2} {vol}_{g} ( G ) . \qquad ( 40 )$$

meaning that it reduces to a volume measure as observed in the smooth case when the pull-back condition is satisfied.

A special case is given when h is the identity, which happens when we embed G into flat Euclidean space. Then

(g*|ᵢ)ⱼₖ= [symbol] ∇f(i,k) [symbol] .

If one considers the gradient flow Eq. (36) coupled with the constraint g=g*(f(t)) for each t, one obtains

$$\frac{d}{dt} E ( f ( t ) , {g}^{*} ( t ) ) = - \frac{1}{2} 〈 {η}_{g}^{- 1} {∇}_{f} E , {∇}_{f} E 〉 \leq 0 ,$$

meaning that one is evolving an initial embedding f₀ towards a stationary configuration—i.e. a harmonic map—of E(f,g*(f)). This represents a generalization of the classi-cal Beltrami flow from images to graphs.

To better understand the graph Beltrami flow, suppose there is node i with neighbors j, k, then

$${η}_{g} ( i ) = \sqrt{{\vert ∇ f ( i , j ) \vert}^{2} {\vert ∇ f ( i , k ) \vert}^{2} - {〈 ∇ f ( i , j ) , ∇ f ( i , k ) 〉}^{2}} ,$$

and the energy along the flow is of the form Eq. (40). It is deduced that, on graphs the discrete Beltrami flow seems to promote gradient alignment.

It is observed that, similarly to the smooth case, when one considers the gradient flow of E with respect to the embedding, one finds in general channel-mixing if h is non-constant. In particular the system of differential equations studied is

∂ₜf^{α}=−½(diag(η_{g}))⁻¹(h|_{f})^{sa}∇_{f*}E(f,g,h).

where the trace of the gradient is taken with respect to a positive definite metric so that the energy along the flow still decreases. One may explicitly write the gradient flow as

$${\partial }_{t} {f}_{i}^{α} = - {Δ}_{g} {f}_{i}^{α} - \frac{{h ⌋}_{f ( i )}^{s α}}{\sqrt{{d}_{i}}} \frac{{η}_{g} ( u )}{2 {η}_{g} ( i )} {( g {\vert}_{u} )}^{jk} ∇ {f}^{β} ( z , k ) {( h {\vert}_{f ( z )} - h {\vert}_{f ( i )} )}_{s β} - \frac{1}{2} {( g {\vert}_{i} )}^{jk} {∇ {f}^{β} ( i , j ) ∇ {f}^{γ} ( i , k ) h ⌋}_{f ( i )}^{s α} {\partial }_{s} {( h {\vert}_{f ( i )} )}_{β γ} . \qquad ( 41 )$$

It is noted that similarly to the smooth case we now have mixing of the channels with terms depending on the derivative of h and hence on the connection coefficients.

When an embedding f₀:(V,g)→ [symbol] ^{d} is considered, encoding both positional and feature information, inspired by the harmonic map flow one may study the gradient flow

∂ₜf^{α}=−Δ_{g}f^{α}; f(0)=f₀,

for 1≤α≤d. When one takes g to be diagonal, one may express the system of differential equations as

$${\partial }_{t} {f}_{i}^{α} = \frac{1}{\sqrt{{d}_{i}}} {\sum }_{j ∼ i} {a}_{g} ( i , j ) {( ∇ f )}^{α} ( i , j ) \qquad ( 42 )$$

$$= div ( diag ( {a}_{g} ) ∇ {f}^{α} ) ( i ) , with \qquad ( 43 )$$

$${a}_{g} ( i , j ) ≡ {η}_{g} ( i ) {( g {\vert}_{i} )}^{jj} + {η}_{g} ( j ) {( g {\vert}_{j} )}^{ii} . \qquad ( 44 )$$

Therefore, the diffusion equation above yields an attention mechanism without mixing of the channels. Since one may have the degrees of freedom provided by the choice of the metric g, this problem may result in a discrete counterpart to the Perona-Malik flow discussed previously. By optimizing with respect to the metric g, one is learning the best non-linear Laplacian Δ_{g} and hence the best diffusivity (attention) coefficients. The caveat of this approach—exactly as for the case of Perona-Malik in image processing—is that in general, for arbitrary choices of g, we might no longer control the sign of E along the evolution equation. This might be beneficial though, since it has been already mentioned that for fixed g the only stationary points of E are the trivial harmonic maps living in the kernel of the normalized Laplacian.

It has al-ready been considered in Riemannian geometry coupling the harmonic map flow with an evolution flow of the underlying Riemannian structure g_{M} on M. This leads to a system of equations of the form

∂ₜf^{α}=Δ_{g}_{ₜ}f^{α}+^{h}Γ_{βγ}^{α}∂ⱼf^{β}∂ₖf^{γ}gₜ^{jk}, (45)

∂ₜg=Φ(gₜ,f). (46)

One sees how the coupled flows mean that the diffusivity terms in the equations for f now explicitly depend on time according to a flow of the underlying geometry.

Similar to the smooth case, flow on the metric structure may be considered as follows.

∂ₜ(g)=Φ(g,t,f).

This can be a diffusion-like flow on the metric structure-acting for example as some metric version of DIGL. It is also noted that while at this level one deals with the metric structure of the graph, one may also use the metric at each time to induce a rewiring—now depending on both topology and embedding (hence features). One then may study the time-dependent energy E[f,g(t),h] and consider its gradient flow

$$\frac{\partial f}{\partial t} ( t ) = - {∇}_{f} E [ f , g ( t ) , h ] .$$

For example, in the case of embedding into Euclidean space (h=I), we find (for a diagonal g)

$$\frac{\partial f}{\partial t} ( t ) = \frac{1}{\sqrt{{d}_{i}}} {\sum }_{j ∼ i} {a}_{g ( t )} ( i , j ) ( ∇ f ) ( i , j ) \qquad ( 47 )$$

$$= \frac{1}{\sqrt{{d}_{i}}} {\sum }_{j ∼ i} a ( i , j , t ) ( ∇ f ) ( i , j ) . \qquad ( 48 )$$

Remark 11. In line with the over-squashing issue, one might consider a diffusion process at the metric level amounting to a feature-aware homogenization. One may then use that to rewire the graph at different times. It is noted that an example of diffusion at the metric level might be given by the Ricci flow, with curvature depending on the features as well.

III. Sheaf Diffusion

There are generalizations to sheaves and arbitrary vector fields. This is to deal better with heterophily and the over-smoothing issue. Namely, one generalizes the definition above to replace the notion of standard gradient with that of derivative induced by the sheaf structure (or modified by a (family of) vector field(s)). This would help in studying an energy map whose minimization may occur at heterophilic embeddings thanks to the potential disagreement of the restriction maps.

A cellular sheaf (e.g., sheaf structure 133 of FIG. 1A) over a graph (e.g., graph 110 of FIGS. 1A and 1B) is a mathematical object associating a space with each node and edge in the graph and a map between these spaces for each incident node-edge pair. A cellular sheaf is defined as follows.

Definition 1. A cellular sheaf (G,F) on an undirected graph G=(V,E) consists of the following:
A vector space F(v) for each v∈V.
A vector space F(e) for each e∈E.
A linear map [symbol] F(v)→F(e) for each incident v [symbol] e node-edge pair.
   One refers to the vector spaces associated with the nodes and edges as stalks, while the linear maps are commonly referred to as restriction maps.

The space formed by all the spaces associated to the nodes of the graph is called the space of 0-cochains and is denoted by C⁰(G; F). Similarly, C¹(G; F)—the space of 1-cochains—contains the data associated with all the edges of the graph.

Definition 2. For a sheaf (G,F) the space of 0-cochains C⁰(G;F)≡⊕_{v∈V} F(v) and 1-cochains C¹(G;F)≡⊕_{e∈E} F(e).

For a 0-cochain x∈C⁰(G; F), one uses xᵥ to refer to the vector in F(v) of node v and similarly for 1-cochains. From an opinion dynamics perspective, xᵥ can be thought of as the private opinion of node v, while [symbol] expressed how that opinion manifests publicly in a discourse space formed by F(e). It is natural to define a linear co-boundary map δ between C⁰ (G; F) and C¹(G; F) which measures the disagreement between all nodes in the discourse space.

Definition 3. For some arbitrary choice of orientation for each edge e=u→v∈E,δ: C⁰(G;F)→C¹(G;F), δ(x)ₑ≡ [symbol] xᵥ− [symbol] xᵤ.

Given a cellular sheaf (G,F), using the co-boundary operator δ, one can define a Sheaf Laplacian operator associated with a sheaf.

Definition 4. The sheaf Laplacian of a sheaf (G,F) is a map L_{F}:C⁰(G;F)→C⁰(G;F) given by L_{F}≡δ^{T} δ.

$${{L}_{F} ( x )}_{v} ≡ {\sum }_{v , u ⊴ e} {F}_{v ⊴ e}^{T} ( {F}_{v ⊴ e} {x}_{v} - {F}_{u ⊴ e} {x}_{u} ) .$$

The sheaf Laplacian is a positive semi-definite block matrix. The diagonal blocks are L_{Fvv}= [symbol] , while the non-diagonal blocks are L_{Fuu}=− [symbol] .

Let G=(V,E) be a graph and consider that all nodes have features that are d-dimensional vectors xᵥ∈F(v). The features of all nodes are represented as a single vector x∈C⁰(G;F) stacking all the individual d-dimensional vectors. Additionally, if there are f feature channels, they can be represented as a matrix X∈R^{(nd)}×f, whose columns are vectors in C⁰(G;F).

The following represents a spatially discretized sheaf diffusion process governed by the following PDE:

X(0)=X, \dot{X}(t)=−Δ_{F}X(t). (49)

It can be shown that in the time limit, each feature channel is projected into the harmonic space of the sheaf Laplacian ker(Δ_{F}).

When considering a discrete, parametric and non-linear version of this process, one may determine how much the weights can steer the process. This may be relevant if the underlying sheaf is only approximately correct for the task to be solved.

The continuous diffusion process from Eq. (49) has the following Euler discretization with unit step-size:

X(t+1)=X(t)−Δ_{F}X(t)=(I_{nd}−Δ_{F})X(t). (50)

Assuming X∈R^{nd×f}^{₁}, one may equip the right side with weight matrices W₁∈R^{d×d}, W₂ ∈R^{f}^{₁}^{×f}^{₂}, and a nonlinearity σ to arrive at the following model:

Y=σ((I_{nd}−Δ_{F})(Iₙ⊗W₁)XW₂)∈R^{nd×f}^{₂}, (51)

where f₁, f₂ are the number of input and output feature channels, and ⊗ denotes the Kronecker product. Here, W₁ multiplies from the left the vector feature of all the nodes in all channels (i.e. W₁xᵥⁱ for all v and channels i), while W₂ multiplies the features from the right and can adjust the number of feature channels, as in GCNs.

There are various advantages one obtains from using the right sheaf-structure for a particular node classification task. However, in general, this ground truth sheaf is unknown or unspecified. Therefore, it is an aim to learn the underlying sheaf from data.

Consider the following diffusion-type equation, which contains the sheaf diffusion equation as a particular case.

\dot{X}(t)=−σ(Δ_{F(t)}(Iₙ⊗W₁)XW₂), (52)

The sheaf Laplacian Δ_{F(t)} is that of a sheaf (G,F(t)) that evolves over time. More specifically, the evolution of the sheaf structure is described by a learnable function of the data (G,F(t))=g(G,X(t)); θ).

A discrete version of this equation may be considered, using a new set of weights at each layer t.

Xₜ₊₁=Xₜ−σ(Δ_{F(t)}(Iₙ⊗W₁^{t})XₜW₂^{t}), (53)

For both models an initial multilayer perceptron (MLP) is used to compute X(0) from the raw features and a final linear layer to perform the node classification. Overall, this represents an entirely new framework for learning on graphs, which does not only evolve the features at each new layer, but also evolves the underlying ‘geometry’ of the graph (i.e., the sheaf structure).

An advantage of learning a sheaf is that one does not require any sort of embedding of the nodes in an ambient space. Instead, everything regarding a sheaf can be learned locally. Each d×d matrix [symbol] is learned via a parametric function Φ:R^{d×2}→R^{d×d}:

[symbol]

_{≡(v,u)}=Φ(xᵥ,xᵤ) (54)

For simplicity, the equation above uses a single feature channel, but in practice, all channels are supplied as input. This function retains the inductive bias of locality specific to GNNs since it only utilizes the features of the nodes forming the edge. At the same time, it is important that this function is non-symmetric in order to be able to learn asymmetric transport maps along each edge. In what follows, one distinguishes between several types of functions Φ depending on the type of matrix they learn.
Diagonal: One of the advantages of this parametrization is that fewer parameters need to be learned per edge and the sheaf Laplacian ends up being a matrix with diagonal blocks, which also results in fewer operations in sparse matrix multiplications. In some examples, the d dimensions of the stalks do not interact.
Orthogonal: In this case, the model effectively learns a discrete vector bundle. Orthogonal matrices provide several advantages: (1) they are able to mix the various dimension of the stalks, (2) the orthogonality constraint prevents over-fitting while reducing the number of parameters, (3) they have better understood theoretical properties, and (4) the resulting Laplacians are easier to normalize numerically since the diagonal entries correspond to the degrees of the nodes. In some implementations, orthogonal matrices are built from a composition of Householder reflections.
General: Finally, one may consider the most general option of learning arbitrary matrices. The maximal flexibility provided by these maps can be useful, but it also comes with the danger of overfitting. At the same time, the sheaf Laplacian is more challenging to normalize numerically since one has to compute D^{-1/2} for a positive semi-definite matrix D. To perform this at scale, one may rely on SVD, whose gradients can be infinite if D has repeated eigenvalues. Therefore, this model is more challenging to train.

The techniques described herein consider representations of diffusion operators on graphs, such as the Laplace-Beltrami operator. The above-discussed techniques need not be limited to the approximation of continuous diffusion operators. Any partial differential operator may be considered in using a GNN to model the evolution of information within a graph. For example, rather than the diffusion equation, variants of the wave equation may be used. The details for deriving a discretized wave operator on a graph may be defined in an analogous fashion as the diffusion operator.

Although the disclosed concepts include those defined in the attached claims, it should be understood that the concepts can also be defined in accordance with the following examples.

Example 1 is a method comprising: obtaining graph data representing a first graph, the first graph including a plurality of nodes and a plurality of edges connecting pairs of nodes of the plurality of nodes, one or more of the plurality of nodes having a respective set of feature coordinates representing a set of features and/or a set of positional coordinates representing a set of positions; inputting the graph data into a graph neural network (GNN) model, the GNN model having an architecture that is based on a discretization scheme for solving a continuous differential equation governing a behavior of the set of feature coordinates and set of positional coordinates of each of the plurality of nodes over space and time; and generating, as output of the GNN model, a respective new set of feature coordinates and/or a respective new set of positional coordinates for at least one of the plurality of nodes.

Example 2 is the method of Example 1, wherein the plurality of nodes represent users of a social network.

Example 3 is the method of any of Examples 1 to 2, wherein the first graph represents at least a portion of a social network.

Example 4 is the method of any of Examples 1 to 3, wherein the architecture of the GNN model includes a plurality of layers.

Example 5 is the method of Example 4, wherein the architecture of the GNN model is based on the discretization scheme for solving the continuous diffusion equation being defined in a metric space governing a behavior of the set of feature coordinates and/or set of positional coordinates of at least one of the plurality of nodes over space and time.

Example 6 is the method of any of Examples 1 to 5, wherein the continuous diffusion equation is defined by a diffusion kernel.

Example 7 is the method of Example 6, wherein the diffusion kernel is based on an optimization of an action functional with respect to the set of feature coordinates and/or set of positional coordinates of the at least one of the plurality of nodes and a metric defined by the metric space.

Example 8 is the method of any of Examples 6 to 7, wherein the diffusion kernel, when discretized according to the discretization scheme, includes a first learnable function of sets of feature coordinates and/or sets of positional coordinates of a pair of nodes of the plurality of nodes.

Example 9 is the method of Example 8, wherein the first learnable function includes an attention matrix representing a diffusivity controlling a diffusion strength between pairs of nodes of the plurality of nodes.

Example 10 is the method of Example 9, wherein the attention matrix is independent of time.

Example 11 is the method of Example 9, wherein the attention matrix is normalized such that a sum of elements of the attention matrix over nodes of the set of nodes sharing an edge of the set of edges with another node of the set of nodes is equal to unity

Example 12 is the method of Example 9, wherein the attention function includes a scaled dot product attention function.

Example 13 is the method of Example 12, wherein the scaled dot product attention function is based on a pair of learned matrices.

Example 14 is the method of any of Examples 7 to 13, wherein the metric defined by the metric space is a pullback of a mapping between a manifold defined within the metric space and another metric space.

Example 15 is the method of any of Examples 1 to 14, wherein the graph data includes a plurality of channels, each of the plurality of channels representing a combination of feature coordinates of the set of feature coordinates.

Example 16 is the method of Example 14, wherein the another metric space is a Euclidean space.

Example 17 is the method of any of Examples 15-16, wherein there is no mixing between channels of the plurality of channels.

Example 18 is the method of any of Examples 14 to 17, wherein the mapping is a harmonic mapping.

Example 19 is the method of Example 18, wherein the harmonic mapping is a stationary point of a Dirichlet energy associated with the mapping.

Example 20 is the method of any of Examples 7 to 19, wherein the metric is time-dependent.

Example 21 is the method of any of Examples 7 to 20, wherein the metric has a flow governed by a function of the metric defined by the metric space and the mapping.

Example 22 is the method of any of Examples 7 to 21, wherein the continuous diffusion equation includes a channel-mixing term dependent on the metric.

Example 23 is the method of any of Examples 1 to 22, wherein the set of feature coordinates and set of positional coordinates of each of the plurality of nodes form a first embedding vector.

Example 24 is the method of Example 23, wherein the continuous differential equation governs a behavior of the first embedding vector.

Example 25 is the method of any of Examples 1 to 24, wherein the new set of feature coordinates and new set of positional coordinates form a second embedding vector.

Example 26 is the method of any of Examples 1 to 25, wherein the set of feature coordinates and set of positional coordinates of each of the plurality of nodes form respective stalks of a first cellular sheaf.

Example 27 is the method of Example 26, wherein the differential equation governs a behavior of the first cellular sheaf.

Example 28 is the method of any of Examples 26 to 27, wherein the new set of feature coordinates and new set of positional coordinates form a second cellular sheaf.

Example 29 is the method of any of Examples 1 to 28, wherein the second embedding vector for each of the plurality of nodes is generated via a second learnable function of the respective set of features and the respective set of positions of each of the plurality of nodes.

Example 30 is the method of any of Examples 1 to 29, wherein the respective new set of feature coordinates for each of the plurality of nodes results in a labeling of the users of the social network represented by the plurality of nodes.

Example 31 is the method of any of Examples 1 to 30, wherein the respective new set of positional coordinates results in a rewiring of the first graph.

Example 32 is a computing system comprising one or more computers and one or more storage devices storing instructions that are operable, when executed by the one or more computers, to cause the one or more computers to perform the method of any of Examples 1 to 31.

Example 33 is a computer storage medium encoded with a computer program, the program comprising instructions that are operable, when executed by a data processing apparatus, to cause the data processing apparatus to perform the method of any of Examples 1 to 31.

Example 34 is an apparatus comprising: a means for performing the method of any of Examples 1 to 31.

Example 35 is a computer program product comprising a non-transitive storage medium, the computer program product including code that, when executed by processing circuitry, causes the processing circuitry to perform a method, the method comprising: obtaining graph data representing a first graph, the first graph representing a social network and having (i) a plurality of nodes representing users of the social network and (ii) a plurality of edges connecting pairs of nodes of the plurality of nodes and representing connections between the users of the social network, each of the plurality of nodes having a respective set of feature coordinates representing a set of features and a set of positional coordinates representing a set of positions, the set of feature coordinates and the set of positional coordinates defining a first embedding vector; inputting the graph data into a graph neural network (GNN) model; and producing, as output of the GNN model, a second embedding vector for at least one of the set of nodes, the second embedding vector for each of the set of nodes resulting in a labeling of the users of the social network represented by the plurality of nodes and a rewiring of the first graph.

Example 36 is the computer program product of Example 35, wherein the GNN model has an architecture including a plurality of layers.

Example 36 is the computer program product of any of Examples 35 to 36, wherein the architecture of the GNN model being is based on a discretization scheme for solving a continuous diffusion equation defined in a metric space governing a behavior of the first embedding vector over space and time and being defined by a diffusion kernel.

Example 37 is the computer program product of Example 36, wherein the diffusion kernel is based on an optimization of an action functional with respect to the embedding vector and a metric defined by the metric space.

Example 38 is the computer program product of any of Examples 36-37, wherein the discretization scheme for solving the continuous diffusion equation includes an approximation of temporal derivatives of the first embedding vector.

Example 39 is the computer program product of Example 38, wherein the approximation is of order at least four.

Example 40 is the computer program product of any of Examples 38 to 39, wherein the approximation is included in a Dormand-Prince discretization scheme.

Example 41 is the computer program product of any of Examples 36-40, wherein the discretization scheme for solving the continuous diffusion equation includes a moving mesh method of approximating spatial derivatives of the first embedding vector.

Example 42 is the computer program product of Example 41, wherein the spatial derivatives include any of derivatives with respect to feature coordinates of the set of feature coordinates and derivatives with respect to positional coordinates of the set of positional coordinates.

Example 43 is the computer program product of any of Examples 35 to 42, wherein the set of positional coordinates of the first embedding vector is encoded according to a hyperbolic encoding of the set of positional coordinates into hyperbolic coordinates.

Example 44 is the computer program product of any of Examples 35 to 43, wherein inputting the graph data into the GNN model includes: generating, as an input into the GNN model, an encoded embedding vector, the encoded embedding vector including a first encoding function of the set of positional coordinates of the first embedding vector and a second encoding function of the set of feature coordinates of the first embedding vector.

Example 45 is the computer program product of any of Examples 36 to 44, wherein the diffusion kernel, when discretized according to the discretization scheme, includes a first learnable function of embedding vectors of a pair of nodes of the plurality of nodes.

Example 46 is the computer program product of Example 45, wherein the first learnable function includes an attention matrix representing a diffusivity controlling a diffusion strength between pairs of nodes of the set of nodes.

Example 47 is the computer program product of Example 46, wherein the attention matrix is independent of time.

Example 48 is the computer program product of any of Examples 46 to 47, wherein the attention matrix is normalized such that a sum of attention matrix elements over all nodes of the set of nodes sharing an edge of the set of edges with another node of the set of nodes is equal to unity.

Example 49 is the computer program product of any of Examples 46 to 48, wherein the attention function includes a scaled dot product attention function.

Example 50 is the computer program product of Example 49, wherein the scaled dot product attention function is based on a pair of learned matrices.

Example 51 is the computer program product of any of Examples 35 to 50, wherein the second embedding vector for each of the plurality of nodes is generated via a second learnable function of the respective set of features and the respective set of positions of each of the plurality of nodes.

Example 52 is a method for performing any of the steps of Examples 35 to 51.

Example 53 is a computing system comprising one or more computers and one or more storage devices storing instructions that are operable, when executed by the one or more computers, to cause the one or more computers to perform the method of any of Examples 35 to 51.

Example 54 is an apparatus comprising a means for performing any of Examples 35 to 51.

Example 55 is a a method, comprising: obtaining graph data representing a first graph, the first graph including a plurality of nodes and a plurality of edges connecting pairs of nodes of the plurality of nodes, each of at least a portion of the plurality of nodes having a first set of features; applying, by a graph neural network (GNN) model, a diffusion process to the first graph to update the first set of features to a second set of features; and generating, as output of the GNN model, a second graph based on the second set of features for each of at least the portion of the plurality of nodes.

Example 56 is the method of Example 55, wherein the diffusion process is based on a set of parameters.

Example 57 is the method of any of Examples 55 to 56, wherein the set of parameters are learned using a learning loss function.

Example 58 is the method of any of Examples 55 to 57, wherein the first graph has a cellular sheaf structure.

Example 59 is the method of any of Examples 55 to 58, wherein the diffusion process includes a sheaf diffusion.

Example 60 is the method of any of Examples 58-59, wherein the cellular sheaf structure is learnable using a learning loss function.

Example 61 is the method of any of Examples 55 to 60, wherein the diffusion process is defined by a differential equation governing a behavior of the update to the second set of features.

Example 62 is the method of any of Examples 55 to 61, wherein applying the diffusion process includes performing a discretization of the differential equation using a numerical discretization scheme.

Example 63 is the method of any of Examples 55 to 62, wherein each of at least the portion of the plurality of nodes further includes positional coordinates of a position of a respective node.

Example 64 is the method of Example 63, wherein applying the diffusion process includes applying the diffusion process on the graph to update the positional coordinates of each of at least the portion of the plurality of nodes.

Example 65 is the method of Example 64, wherein applying the diffusion process includes performing a rewiring operation on the first graph.

Example 66 is a computing system comprising one or more computers and one or more storage devices storing instructions that are operable, when executed by the one or more computers, to cause the one or more computers to perform the method of any of Examples 55 to 65.

Example 67 is a computer storage medium encoded with a computer program, the program comprising instructions that are operable, when executed by a data processing apparatus, to cause the data processing apparatus to perform the method of any of Examples 55 to 65.

Example 68 is an apparatus comprising: a means for performing the method of any of Examples 55 to 65.

Example 69 is a method, comprising: obtaining graph data representing a first graph, the first graph including a plurality of nodes and a plurality of edges connecting pairs of nodes of the plurality of nodes, each of at least a portion of the plurality of nodes having a first set of features; applying, by a graph neural network (GNN) model, a node diffusion process to the first graph to update the first set of features to a second set of features; applying a graph diffusion process to the first graph to update the first graph, the graph diffusion process being coupled to the node diffusion process; and generating, as output of the GNN model, a second graph based on the second set of features for each of at least the portion of the plurality of nodes.

Example 70 is the method of Example 69, wherein the graph diffusion process is based on a discrete curvature of the first graph.

Example 71 is the method of any of Examples 69 to 70, wherein the graph diffusion process is applied before the node diffusion process.

Example 72 is a computing system comprising one or more computers and one or more storage devices storing instructions that are operable, when executed by the one or more computers, to cause the one or more computers to perform the method of any of Examples 69 to 71.

Example 73 is a computer storage medium encoded with a computer program, the program comprising instructions that are operable, when executed by a data processing apparatus, to cause the data processing apparatus to perform the method of any of Examples 69 to 71.

Example 74 is an apparatus comprising: a means for performing the method of any of Examples 69 to 71.

Implementations of the various techniques described herein may be implemented in digital electronic circuitry, or in computer hardware, firmware, software, or in combinations of them. Implementations may be implemented as a computer program product, i.e., a computer program tangibly embodied in an information carrier, e.g., in a machine-readable storage device (computer-readable medium, a non-transitory computer-readable storage medium, a tangible computer-readable storage medium) or in a propagated signal, for processing by, or to control the operation of, data processing apparatus, e.g., a programmable processor, a computer, or multiple computers. A computer program, such as the computer program(s) described above, can be written in any form of programming language, including compiled or interpreted languages, and can be deployed in any form, including as a stand-alone program or as a module, component, subroutine, or other unit suitable for use in a computing environment. A computer program can be deployed to be processed on one computer or on multiple computers at one site or distributed across multiple sites and interconnected by a communication network.

Method steps may be performed by one or more programmable processors executing a computer program to perform functions by operating on input data and generating output. Method steps also may be performed by, and an apparatus may be implemented as, special purpose logic circuitry, e.g., an FPGA (field programmable gate array) or an ASIC (application-specific integrated circuit).

Processors suitable for the processing of a computer program include, by way of example, both general and special purpose microprocessors, and any one or more processors of any kind of digital computer. Generally, a processor will receive instructions and data from a read-only memory or a random access memory or both. Elements of a computer may include at least one processor for executing instructions and one or more memory devices for storing instructions and data. Generally, a computer also may include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in special purpose logic circuitry.

To provide for interaction with a user, implementations may be implemented on a computer having a display device, e.g., a cathode ray tube (CRT) or liquid crystal display (LCD) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Implementations may be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of such back-end, middleware, or front-end components. Components may be interconnected by any form or medium of digital data communication, e.g., a communication network. Examples of communication networks include a local area network (LAN) and a wide area network (WAN), e.g., the Internet.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. A method, comprising:
obtaining graph data representing a first graph, the first graph representing at least a portion of a social network, the first graph including a plurality of nodes representing users of the social network and a plurality of edges connecting pairs of nodes of the plurality of nodes and representing connections between the users of the social network, each of the plurality of nodes having a respective set of feature coordinates representing a set of features and a set of positional coordinates representing a set of positions;
inputting the graph data into a graph neural network (GNN) model, the GNN model having an architecture that is based on a discretization scheme for solving a continuous differential equation governing a behavior of the set of feature coordinates and set of positional coordinates of each of the plurality of nodes over space and time; and
generating, as output of the GNN model, a respective new set of feature coordinates and a respective new set of positional coordinates for at least one of the plurality of nodes.

2. The method as in claim 1, wherein the architecture of the GNN model includes a plurality of layers, the architecture of the GNN model based on the discretization scheme for solving the continuous diffusion equation being defined in a metric space governing a behavior of the set of feature coordinates and set of positional coordinates of at least one of the plurality of nodes over space and time and being defined by a diffusion kernel, the diffusion kernel being based on an optimization of an action functional with respect to the set of feature coordinates and set of positional coordinates of the at least one of the plurality of nodes and a metric defined by the metric space.

3. The method as in claim 2, wherein the diffusion kernel, when discretized according to the discretization scheme, includes a first learnable function of sets of feature coordinates and sets of positional coordinates of a pair of nodes of the plurality of nodes, the first learnable function including an attention matrix representing a diffusivity controlling a diffusion strength between pairs of nodes of the plurality of nodes.

4. The method as in claim 3, wherein the attention matrix is independent of time.

5. The method as in claim 3, wherein the attention matrix is normalized such that a sum of elements of the attention matrix over nodes of the set of nodes sharing an edge of the set of edges with another node of the set of nodes is equal to unity.

6. The method as in claim 3, wherein the attention function includes a scaled dot product attention function, the scaled dot product attention function being based on a pair of learned matrices.

7. The method as in claim 2, wherein the metric defined by the metric space is a pullback of a mapping between a manifold defined within the metric space and another metric space.

8. The method as in claim 7, wherein the graph data includes a plurality of channels, each of the plurality of channels representing a combination of feature coordinates of the set of feature coordinates, and
wherein the another metric space is a Euclidean space, and
wherein there is no mixing between channels of the plurality of channels.

9. The method as in claim 7, wherein the mapping is a harmonic mapping, the harmonic mapping being a stationary point of a Dirichlet energy associated with the mapping.

10. The method as in claim 9, wherein the metric is time-dependent,
wherein the metric has a flow governed by a function of the metric defined by the metric space and the mapping, and
wherein the continuous diffusion equation includes a channel-mixing term dependent on the metric.

11. The method as in claim 1, wherein the set of feature coordinates and set of positional coordinates of each of the plurality of nodes form a first embedding vector,
wherein the continuous differential equation governs a behavior of the first embedding vector, and
wherein the new set of feature coordinates and new set of positional coordinates form a second embedding vector.

12. The method as in claim 1, wherein the set of feature coordinates and set of positional coordinates of each of the plurality of nodes form respective stalks of a first cellular sheaf,
wherein the differential equation governs a behavior of the first cellular sheaf, and
wherein the new set of feature coordinates and new set of positional coordinates form a second cellular sheaf.

13. The method as in claim 11, wherein the second embedding vector for each of the plurality of nodes is generated via a second learnable function of the respective set of features and the respective set of positions of each of the plurality of nodes.

14. The method as in claim 1, wherein the respective new set of feature coordinates for each of the plurality of nodes results in a labeling of the users of the social network represented by the plurality of nodes, and the respective new set of positional coordinates results in a rewiring of the first graph.

15. A computer program product comprising a nontransitive storage medium, the computer program product including code that, when executed by processing circuitry, causes the processing circuitry to perform a method, the method comprising:
obtaining graph data representing a first graph, the first graph representing a social network and having (i) a plurality of nodes representing users of the social network and (ii) a plurality of edges connecting pairs of nodes of the plurality of nodes and representing connections between the users of the social network, each of the plurality of nodes having a respective set of feature coordinates representing a set of features and a set of positional coordinates representing a set of positions, the set of feature coordinates and the set of positional coordinates defining a first embedding vector;
inputting the graph data into a graph neural network (GNN) model; and
producing, as output of the GNN model, a second embedding vector for at least one of the set of nodes, the second embedding vector for each of the set of nodes resulting in a labeling of the users of the social network represented by the plurality of nodes and a rewiring of the first graph.

16. The computer program product as in claim 15, wherein the GNN model has an architecture including a plurality of layers, the architecture of the GNN model being based on a discretization scheme for solving a continuous diffusion equation defined in a metric space governing a behavior of the first embedding vector over space and time and being defined by a diffusion kernel, the diffusion kernel being based on an optimization of an action functional with respect to the embedding vector and a metric defined by the metric space.

17. The computer program product as in claim 16, wherein the discretization scheme for solving the continuous diffusion equation includes an approximation of temporal derivatives of the first embedding vector, the approximation being of order at least four.

18. The computer program product as in claim 17, wherein the approximation is included in a Dormand-Prince discretization scheme.

19. The computer program product as in claim 16, wherein the discretization scheme for solving the continuous diffusion equation includes a moving mesh method of approximating spatial derivatives of the first embedding vector, the spatial derivatives including any of derivatives with respect to feature coordinates of the set of feature coordinates and derivatives with respect to positional coordinates of the set of positional coordinates.

20. The computer program product as in claim 19, wherein the set of positional coordinates of the first embedding vector is encoded according to a hyperbolic encoding of the set of positional coordinates into hyperbolic coordinates.

21. The computer program product as in claim 19, wherein inputting the graph data into the GNN model includes:
generating, as an input into the GNN model, an encoded embedding vector, the encoded embedding vector including a first encoding function of the set of positional coordinates of the first embedding vector and a second encoding function of the set of feature coordinates of the first embedding vector.

22. The computer program product as in claim 16, wherein the diffusion kernel, when discretized according to the discretization scheme, includes a first learnable function of embedding vectors of a pair of nodes of the plurality of nodes, the first learnable function including an attention matrix representing a diffusivity controlling a diffusion strength between pairs of nodes of the set of nodes.

23. The computer program product as in claim 22, wherein the attention matrix is independent of time.

24. The computer program product as in claim 22, wherein the attention matrix is normalized such that a sum of attention matrix elements over all nodes of the set of nodes sharing an edge of the set of edges with another node of the set of nodes is equal to unity.

25. The computer program product as in claim 22, wherein the attention function includes a scaled dot product attention function, the scaled dot product attention function being based on a pair of learned matrices.

26. The computer program product as in claim 15, wherein the second embedding vector for the at least one of the plurality of nodes is generated via a second learnable function of the respective set of features and the respective set of positions of the at least one of the plurality of nodes.

27. A method, comprising:
obtaining graph data representing a first graph, the first graph including a plurality of nodes and a plurality of edges connecting pairs of nodes of the plurality of nodes, each of at least a portion of the plurality of nodes having a first set of features;
applying, by a graph neural network (GNN) model, a diffusion process to the first graph to update the first set of features to a second set of features; and
generating, as output of the GNN model, a second graph based on the second set of features for at least one of at least the portion of the plurality of nodes.

28. The method as in claim 27, wherein the first graph has a cellular sheaf structure, and wherein the diffusion process includes a sheaf diffusion.

29. The method as in claim 28, wherein the cellular sheaf structure is learnable using a learning loss function.

30. The method as in claim 27, wherein the diffusion process is defined by a differential equation governing a behavior of the update to the second set of features.

31. The method as in claim 30, wherein applying the diffusion process includes performing a discretization of the differential equation using a numerical discretization scheme.

32. The method as in claim 27, wherein each of at least the portion of the plurality of nodes further includes positional coordinates of a position of a respective node, and
wherein applying the diffusion process includes applying the diffusion process on the graph to update the positional coordinates of at least the portion of the plurality of nodes.

33. The method as in claim 32, wherein applying the diffusion process includes performing a rewiring operation on the first graph.

34. A method, comprising:
obtaining graph data representing a first graph, the first graph including a plurality of nodes and a plurality of edges connecting pairs of nodes of the plurality of nodes, each of at least a portion of the plurality of nodes having a first set of features;
applying, by a graph neural network (GNN) model, a node diffusion process to the first graph to update the first set of features to a second set of features;
applying a graph diffusion process to the first graph to update the first graph, the graph diffusion process being coupled to the node diffusion process; and
generating, as output of the GNN model, a second graph based on the second set of features for at least one of the portion of the plurality of nodes.

35. The method as in claim 34, wherein the graph diffusion process is based on a discrete curvature of the first graph.
